(19) United States
(12) Patent Application Publication
(10) Pub. No.: US 2016/0106864 A1
(43) Pub. Date: Apr. 21, 2016

(54) CELL PENETRATING PEPTIDE INTRODUCED DRUG-DELIVERY CARRIER COMPRISING MACROMOLECULE

(71) Applicant: AMOREPACIFIC CORPORATION, Seoul (KR)

(72) Inventors: Hyung Jun LIM, Yongin-si (KR); Nok Hyun PARK, Yongin-si (KR); Yuri CHOI, Yongin-si (KR); Il-Hong BAE, Yongin-si (KR); Myeong Jin GOH, Yongin-si (KR); Jae Won YOU, Yongin-si (KR); Song Seok SHIN, Yongin-si (KR); Jon Hwan LEE, Yongin-si (KR)

(21) Appl. No.: 14/883,327

(22) Filed: Oct. 14, 2015

(30) Foreign Application Priority Data
Oct. 15, 2014 (KR) 10-2014-0139024
Sep. 15, 2015 (KR) 10-2015-0130127

(51) Int. Cl. A61K 47/48; A61K 8/64; A61K 9/107; A61Q 19/00; A61K 9/127; A61K 8/06; A61K 9/00; A61K 8/14

(52) U.S. Cl. CPC A61K 47/48815; A61K 9/0014; A61K 8/64; A61K 8/14; A61Q 19/00; A61K 9/1275; A61K 8/062; A61K 9/1075; A61K 9/1271; A61K 2800/74; A61K 2800/654

(57) ABSTRACT

The present disclosure relates to a drug delivery carrier containing a lipid structure or a polymer particle which is covalently bonded to a cell-penetrating AP-GRR peptide (SEQ ID NO 1) or is modified with the peptide chain containing the peptide. The present disclosure also relates to a composition containing the drug delivery carrier and a physiologically active ingredient encapsulated in the carrier. The drug delivery carrier of the present disclosure can effectively deliver macromolecules that are difficult to be delivered into cells, thereby improving the bioavailability of the macromolecules.

### CROSS-REFERENCE TO RELATED APPLICATION

This application claims the priority of Korean Patent Application No. 10-2014-0139024, filed on Oct. 15, 2015, and Korean Patent Application No. 10-2015-0130127, filed on Sep. 15, 2015, and all the benefits accruing therefrom under 35 U.S.C. §119, the contents of which in its entirety are herein incorporated by reference.

### BACKGROUND

1. Field

The present disclosure relates to a cell-penetrating peptide-introduced drug delivery carrier containing a macromolecule.

2. Description of the Related Art

In general, hydrophilic and large substances cannot penetrate into cells through the cell membrane barrier. The cell membrane prevents macromolecules such as peptides, proteins and nucleic acids from entering into cells. Even when they enter into cells via a physiological mechanism called endocytosis by the cell membrane receptor, they are degraded after being fused with the lysosomal compartment. Accordingly, there are many obstacles to treatment and prevention of diseases using them.

Therefore, development of new systems that can effectively deliver a biomolecule into cells and has no cytotoxicity is required and necessary. Several solutions have been presented recently. In particular, cell-permeable peptides are drawing a lot of attentions because they can improve the utility value of macromolecules such as therapeutic proteins and genes that were difficult to be used as drugs due to low cell membrane permeability and short in vivo half-life.

The peptides capable of penetrating into the cell membrane are mainly membrane-penetrating peptides derived from proteins and can be largely classified into three kinds. The first is penetratin, a peptide derived from a homeodomain having an amino acid sequence of SEQ ID NO 2 (Drosophila melanogaster, amino acid sequence: Arg Gln Ile Lys Ile Trp Phe Gln Asn Arg Met Lys Trp Lys Lys). It was found in the homeodomain of Antennapedia, which is the homeoprotein of drosophila (A. Joliot et al., Proc. Natl. Acad. Sci. U.S.A., (1991) 88, 1864). The homeoprotein is a kind of transcription factor and has a structure called the homeodomain consisting of around 60 amino acids that can bind to DNA. The second is the Tat₄₉₋₅₇ peptide located between residues 49-57 of the Tat protein which is a transcription-associated protein of human immunodeficiency virus type 1 (HIV-1) that causes acquired immune deficiency syndrome (AIDS). It has an amino acid sequence of SEQ ID NO 3 (human immunodeficiency virus type 1, amino acid sequence: Arg Lys Lys Arg Arg Gln Arg Arg Arg) (P. A. Wender et al., PNAS (2000) 97, 24, 13003-13008). The third is a peptide based on a membrane translocating sequence (MTS) or a signal sequence. It was found out that it is recognized by an acceptor protein which helps the proteins newly synthesized by RNA to be located on the membrane of an appropriate organelle and that the MTS bound to a nuclear localization signal (NLS) crosses the cell membrane and is accumulated in the cell nucleus in some cell types. This was identified in the MTS derived from the hydrophobic region of the signal sequence in, for example, Kaposi sarcoma fibroblast growth factor 1 (K-FGF), human beta3 integrin, HIV-1 gp41, etc. bound to the NLS peptide derived from nuclear transcription factor kappa B (NF-κB)), simian virus 40 (SV40) T antigen or K-FGF (Y. Lin et al., J. Biol. Chem. (1996) 271, 5305; X. Lin et al., Proc. Natl. Acad. Sci. U.S.A (1996) 93, 11819; M. C. Morris et al. Nucleic Acids Res. (1997) 25, 2730; L. Zhang et al. Proc. Natl. Acad. Sci. U.S.A (1998) 95, 9184; Chaloin et al., Biochiem. Biochim. Res. Commun. (1998) 243, 601; Y. Lin et al., J. Biol. Chem. (1995) 270, 14255).

### REFERENCES OF THE RELATED ART

### Non-Patent Documents

A. Joliot et al., Proc. Natl. Acad. Sci. U.S.A., (1991) 88, 1864.
P. A. Wender et al., PNAS (2000) 97, 24, 13003-13008.
Y. Lin et al., J. Biol. Chem. (1996) 271, 5305.
X. Lin et al., Proc. Natl. Acad. Sci. U.S.A (1996) 93, 11819.
M. C. Morris et al. Nucleic Acids Res. (1997) 25, 2730.
L. Zhang et al. Proc. Natl. Acad. Sci. U.S.A (1998) 95, 9184.
Chaloin et al., Biochiem. Biochim. Res. Commun. (1998) 243, 601.
Y. Lin et al., J. Biol. Chem. (1995) 270, 14255.

### SUMMARY

In order to deliver a macromolecular substance that cannot penetrate into cells easily into cells, the present disclosure is directed to providing a composition containing a cell-penetrating peptide and a physiologically active ingredient.

In an aspect, the present disclosure provides a composition containing a drug delivery carrier containing a lipid structure or a polymer particle covalently bonded to an AP-GRR peptide or a peptide chain containing the same and a physiologically active ingredient encapsulated in the carrier.

The drug delivery carrier according to an aspect of the present disclosure remarkably enhances the delivery of a macromolecular substance having a large molecular weight as a physiologically active ingredient since an AP-GRR peptide capable of effectively increasing membrane permeability is introduced therein. Therefore, the drug delivery carrier according to an aspect of the present disclosure overcomes the drawback that macromolecular physiologically active ingredients such as polysaccharides, enzymes, peptides, drugs, proteins, etc. cannot be derived well into cells.

### BRIEF DESCRIPTION OF THE DRAWINGS

FIG. 1A shows a transmission electron microscopic image of a comparative example.

FIG. 1B shows a transmission electron microscopic image of aqueous liposome solutions containing drug delivery carriers according to the present disclosure.

FIG. 2A shows a result of treating cells with a drug delivery carrier carrying Rhodamine B according to an aspect of the present disclosure and analyzing its amount in the cells by FACS.

FIG. 2B shows a result of treating cells with a drug delivery carrier carrying Dextran-RITC according to an aspect of the present disclosure and analyzing its amount in the cells by FACS.

FIG. 2C shows numerical data of FIG. 2A and FIG. 2B.

FIG. 3 shows a result of treating cells with a drug delivery carrier carrying rhodamine B according to an aspect of the present disclosure and observing the cells with a confocal laser scanning microscope (CLSM).

FIG. 4 shows a result of treating cells with a drug delivery carrier carrying dextran-RITC according to an aspect of the present disclosure and observing the cells with a confocal laser scanning microscope (CLSM).

FIG. 5 shows microscopic images showing a result of absorption of rhodamine B and dextran-RITC in PBS or a general liposome through skin.

FIG. 6 shows a result of microscopic images showing a result of absorption of rhodamine B and dextran-RITC in PBS or a drug delivery carrier according to an aspect of the present disclosure through skin.

FIG. 7A shows a quantitative result of absorption of rhodamine B through skin without stratum corneum.

FIG. 7B shows a quantitative result of absorption of rhodamine B through stratum corneum.

FIG. 8A shows a quantitative result of absorption of dextran-RITC through skin without stratum corneum.

FIG. 8B shows a quantitative result of absorption of dextran-RITC through stratum corneum.

### DETAILED DESCRIPTION

In an aspect, the present disclosure may relate to a drug delivery carrier containing a lipid structure or a polymer particle covalently bonded to a cell-penetrating peptide or a peptide chain containing the same.

In an aspect of the present disclosure, a physiologically active ingredient may be encapsulated in the lipid structure or the polymer particle.

In an aspect of the present disclosure, the lipid structure may be lipid construct.

In an aspect of the present disclosure, the polymer particle may be a polymer structure or a polymer construct.

In an aspect of the present disclosure, the physiologically active ingredient may have a number-average molecular weight or a weight-average molecular weight of 500 Da or greater. Specifically, the physiologically active ingredient may be a water-soluble or water-insoluble macromolecule.

In an aspect of the present disclosure, when the drug delivery carrier contains a lipid structure, the physiologically active ingredient may be a water-soluble macromolecule.

In an aspect of the present disclosure, when the drug delivery carrier contains a polymer particle, the physiologically active ingredient may be a water-insoluble macromolecule.

In an aspect of the present disclosure, the cell-penetrating peptide may be an AP-GRR peptide having a sequence of Gly (Arg)ₙ Gly Tyr Lys Cys (1≦n≦20).

In an aspect of the present disclosure, the n may satisfy 3≦n≦9.

In an aspect of the present disclosure, the cell-penetrating peptide may contain a sequence of SEQ ID NO 1 (amino acid sequence: Gly Arg Arg Arg Arg Arg Arg Arg Arg Arg Gly Tyr Lys Cys). Specifically, in an aspect of the present disclosure, the cell-penetrating peptide may have a sequence of SEQ ID NO 1 (Gly Arg Arg Arg Arg Arg Arg Arg Arg Arg Gly Tyr Lys Cys).

In an aspect of the present disclosure, the lipid structure or the polymer particle may contain an amphiphilic polymer.

In an aspect of the present disclosure, when the polymer particle further contains an amphiphilic polymer, the polymer particle may be an amphiphilic polymer or may be prepared therefrom.

In an aspect of the present disclosure, the cell-penetrating peptide or the peptide chain containing the same may be covalently bonded to the amphiphilic polymer.

In an aspect of the present disclosure, the covalent boding between the cell-penetrating peptide or the peptide chain containing the same and the lipid structure or the polymer particle may be a covalent boding formed between a maleimide group and a thiol group. It is known to those skilled in the art that such a covalent boding is fairly stable.

In an aspect of the present disclosure, the cell-penetrating peptide or the peptide chain containing the same may be bonded to the lipid structure, the polymer particle or the amphiphilic polymer contained therein through a bonding between a maleimide group and a thiol group, although not being limited thereto as long as a stable covalent boding can be formed. Since the cell-penetrating peptide according to an aspect of the present disclosure has a thiol group, a maleimide group may be introduced into the lipid structure, the polymer particle or the amphiphilic polymer contained therein to induce a covalent boding with the cell-penetrating peptide. In an aspect of the present disclosure, a maleimide group may be introduced into the lipid structure, the polymer particle or the amphiphilic polymer contained therein by binding a maleimide group to a carboxyl group of the lipid structure, the polymer particle or the amphiphilic polymer contained therein.

In an aspect of the present disclosure, the amphiphilic polymer may be one or more selected from a group consisting of an alkylated hyaluronic acid having an alkyl group attached to a hyaluronic acid side chain, a poly(methacrylic acid-co-n-alkyl methacrylate) random copolymer of Chemical Formula 1 and a poly(hydroxyethyl methacrylate-co-n-alkyl methacrylate) random copolymer of Chemical Formula 2, although not being limited thereto. In an aspect of the present disclosure, the amphiphilic polymer may be a general acrylate-based polymer prepared from polymerization by a general free radical thermal initiation method.

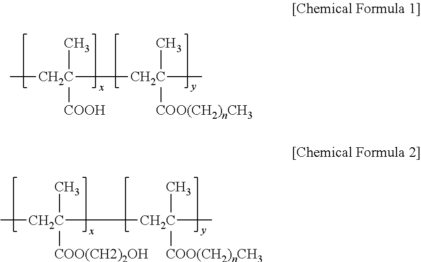

In Chemical Formula 1 and 2,

7≦n≦22, and

a molar ratio of x:y is from 90:10 to 50:50.

In an aspect of the present disclosure, the poly(methacrylic acid-co-n-alkyl methacrylate) random copolymer may consist of two monomers methacrylic acid and n-alkyl methacrylate. The polymer may be prepared from polymerization by a general free radical thermal initiation method. Also, anionic or cationic polymerization may be employed for control of molecular weight distribution.

In an aspect of the present disclosure, the poly(hydroxyethyl methacrylate-co-n-alkyl methacrylate) random copolymer may consist of two monomers hydroxyethyl methacrylate and n-alkyl methacrylate. The polymer may be prepared from polymerization by a general free radical thermal initiation method. Also, anionic or cationic polymerization may be employed for control of molecular weight distribution.

In an aspect of the present disclosure, in Chemical Formulas 1 and 2, the n may be an integer from 5 to 30, specifically an integer from 7 to 22, more specifically an integer from 11 to 22.

In an aspect of the present disclosure, in Chemical Formulas 1 and 2, a molar ratio of x:y may be an integer from 50 to 90:an integer from 10 to 50, specifically 90:10, 85:15, 70:30, 60:40 or 50:50. Specifically, it may be from 85:15 to 70:30.

In an aspect of the present disclosure, the molecular weight of the amphiphilic polymer having a structure of Chemical Formula 1 or 2 affects the structure of a polymer-liposome nanocomposite. The polymer used in the drug delivery carrier according to an aspect of the present disclosure may have a number-average molecular weight or 5,000-100,000, specifically 10,000-50,000.

In the drug delivery carrier according to an aspect of the present disclosure, the lipid structure may have a more stable structure because of the amphiphilic polymer.

For a lipid-cholesterol-based liposome to be used for cosmetics and agents for external application to skin, stability should be ensured in the formulation. However, it easily loses its structure due to various surfactants present in the formulation. The disadvantage of structural instability may be overcome to some extent by introducing an amphiphilic polymer into the liposome. The amphiphilic polymer-introduced polymer-liposome composite maintains a shape similar to that of the lipid-cholesterol-based liposome and has a structure wherein the hydrophobic moiety of the amphiphilic polymer is assembled between the lipid-cholesterol-based lipid bilayer, tightly binding the lipid bilayer thereby protecting the outer wall and stably maintaining the structure of the liposome against various factors making the liposome structure unstable such as salts, surfactants, etc.

In the drug delivery carrier according to an aspect of the present disclosure, the polymer that may be used as the amphiphilic polymer may have a number-average molecular weight of 5,000-200,000 Da, specifically 10,000-100,000 Da. The amphiphilic polymer may have a number-average molecular weight of 1,000 Da or greater, 2,000 Da or greater, 3,000 Da or greater, 4,000 Da or greater, 5,000 Da or greater, 6,000 Da or greater, 7,000 Da or greater, 8,000 Da or greater, 9,000 Da or greater, 10,000 Da or greater, 11,000 Da or greater, 12,000 Da or greater, 13,000 Da or greater, 14,000 Da or greater, 15,000 Da or greater, 20,000 Da or greater, 30,000 Da or greater, 50,000 Da or greater or 100,000 Da or greater, or 200,000 Da or smaller, 150,000 Da or smaller, 100,000 Da or smaller, 90,000 Da or smaller, 80,000 Da or smaller, 70,000 Da or smaller, 60,000 Da or smaller, 50,000 Da or smaller, 40,000 Da or smaller, 30,000 Da or smaller, 20,000 Da or smaller, 10,000 Da or smaller, 5,000 Da or smaller, 3,000 Da or smaller or 1,000 Da or smaller, although not being limited thereto.

In an aspect of the present disclosure, the amphiphilic polymer may have a molar ratio of a hydrophobic moiety to a hydrophilic moiety of 10-50%. When the molar ratio is smaller than 10%, the polymer may be partly present in an aqueous phase independently and may act as a surfactant. And, when it exceeds 50%, i.e. when the hydrophobic moiety of the polymer is dominant, the structure of the liposome in the composite with the liposome may become unstable.

In an aspect of the present disclosure, the amphiphilic polymer may be prepared from polymerization by a general free radical thermal initiation method. Also, anionic or cationic polymerization may be employed for control of molecular weight distribution. In addition, for a natural polymer such as hyaluronic acid, an alkyl chain may be covalently bonded to a side chain to confer hydrophobicity.

In an aspect of the present disclosure, a weight ratio of the lipid structure or the polymer particle:the amphiphilic polymer may be 50-99 wt %:1-50 wt %, specifically 70-90 wt %:10-30 wt %, based on the total weight of a mixture thereof, and the lipid structure may contain cholesterol.

When the drug delivery carrier of the present disclosure further contains the amphiphilic polymer to enhance the structural stability of the liposome, the amphiphilic polymer may be contained in an amount of 1-50 wt %, specifically 10-30 wt %, based on the total weight of the mixture of the lipid structure or the polymer particle and the amphiphilic polymer. At this ratio, it is easy to prepare the most stable polymer-liposome composite.

In an aspect of the present disclosure, the “number-average molecular weight” may mean an average molecular weight obtained by averaging the molecular weight of a molecular species of a polymer compound having a molecular weight distribution with a number fraction or mole fraction, and the “weight-average molecular weight” may mean an average molecular weight obtained by averaging the molecular weight of a molecular species of a polymer compound having a molecular weight distribution with a weight fraction. The number-average molecular weight and the weight-average molecular weight may be calculated by methods obvious to those skilled in the art to which the present disclosure belongs.

In an aspect of the present disclosure, the physiologically active ingredient may have a number-average molecular weight or a weight-average molecular weight of 1,000 Da or greater, 2,000 Da or greater, 3,000 Da or greater, 4,000 Da or greater, 5,000 Da or greater, 6,000 Da or greater, 7,000 Da or greater, 8,000 Da or greater, 9,000 Da or greater, 10,000 Da or greater, 11,000 Da or greater, 12,000 Da or greater, 13,000 Da or greater, 14,000 Da or greater, 15,000 Da or greater, 20,000 Da or greater, 30,000 Da or greater, 50,000 Da or greater, 100,000 Da or greater, 300,000 Da or greater, 500,000 Da or 1,000,000 Da or greater, or 5,000,000 Da or smaller, 4,000,000 Da or smaller, 3,000,000 Da or smaller, 2,000,000 Da or smaller or 1,000,000 Da or smaller, although not being limited thereto.

In an aspect of the present disclosure, the physiologically active ingredient may be one or more selected from a group consisting of a synthesized water-soluble macromolecular substance, a macromolecular substance obtained by extraction from a natural product, an enzyme, EGF (epidermal growth factor), a protein, a peptide and a polysaccharide as a macromolecule. The physiologically active ingredient may be one exhibiting useful skin-moisturizing, skin-whitening or antioxidant effects.

In an aspect of the present disclosure, the physiologically active ingredient may be a water-soluble macromolecule in the broadest concept, including synthetized, extracted or naturally occurring water-soluble macromolecules. The macromolecule may be one providing useful effects on skin.

In an aspect, the present disclosure may relate to a composition containing the drug delivery carrier according to an aspect of the present disclosure and a physiologically active ingredient encapsulated in the carrier.

In an aspect of the present disclosure, the composition may be a pharmaceutical composition or a cosmetic composition.

In an aspect of the present disclosure, the formulation of the cosmetic composition is not particularly limited and may be selected adequately depending on purposes. For example, it may be prepared into one or more formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser, although not being limited thereto.

When the cosmetic composition according to an aspect of the present disclosure is formulated as a paste, a cream or a gel, animal fiber, plant fiber, wax, paraffin, starch, tragacanth, cellulose derivatives, polyethylene glycol, silicone, bentonite, silica, talc, zinc oxide, etc. may be used as a carrier component.

When the cosmetic composition according to an aspect of the present disclosure is formulated as a powder or a spray, lactose, talc, silica, aluminum hydroxide, calcium silicate or polyamide powder may be used as a carrier component. In particular, when the formulation is a spray, it may further contain a propellant such as chlorofluorohydrocarbon, propane/butane or dimethyl ether.

When the cosmetic composition according to an aspect of the present disclosure is formulated as a solution or an emulsion, a solvent, a solvating agent or an emulsifier may be used as a carrier component. For example, water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan may be used.

When the cosmetic composition according to an aspect of the present disclosure is formulated as a suspension, a liquid diluent such as water, ethanol or propylene glycol, a suspending agent such as ethoxylated isostearyl alcohol, polyoxyethylene sorbitol ester and polyoxyethylene sorbitan ester, microcrystalline cellulose, aluminum metahydroxide, bentonite, agar, tragacanth, etc. may be used as a carrier component.

When the cosmetic composition according to an aspect of the present disclosure is a surfactant-containing cleanser, aliphatic alcohol sulfate, aliphatic alcohol ether sulfate, sulfosuccinic acid monoester, isethionate, imidazolinium derivatives, methyl taurate, sarcosinate, fatty acid amide ether sulfate, alkylamidobetaine, aliphatic alcohol, fatty acid glyceride, fatty acid diethanolamide, vegetable oil, lanolin derivative, ethoxylated glycerol fatty acid ester, etc. may be used as a carrier component.

The cosmetic composition according to an aspect of the present disclosure may further contain a functional additive and ingredients contained in general cosmetic compositions. The functional additive may include one or more ingredient selected from a group consisting of a water-soluble vitamin, an oil-soluble vitamin, a polypeptide, a polysaccharide, a sphingolipid and a seaweed extract. These functional additives are physiologically active ingredients and may be encapsulated in the drug delivery carrier according to an aspect of the present disclosure.

In an aspect of the present disclosure, the cosmetic composition may further contain, together with the functional additive, ingredients contained in general cosmetic compositions, if necessary. The additionally contained ingredient may include an oil, a fat, a humectant, an emollient, a surfactant, an organic or inorganic pigment, an organic powder, a UV absorbent, an antiseptic, a sterilizer, an antioxidant, a plant extract, a pH control agent, an alcohol, a colorant, a fragrance, a blood circulation accelerator, a coolant, a deodorant, purified water, etc.

The pharmaceutical composition according to an aspect of the present disclosure may be prepared into various formulations for oral or parenteral administration. A diluent or an excipient such as a filler, an extender, a binder, a wetting agent, a disintegrant, surfactant, etc. is commonly used to prepare the formulation. Solid formulations for oral administration include a tablet, a pill, a powder, a granule, a soft or hard capsule, etc. The solid formulation is prepared mixing with at least one excipient, for example, starch, calcium carbonate, sucrose, lactose, gelatin, etc. In addition to the simple excipient, a lubricant such as magnesium stearate, talc, etc. is also used. Liquid formulations for oral administration include a suspension, a solution for internal use, an emulsion, a syrup, etc. In addition to a commonly used simple diluent such as water and liquid paraffin, various excipients, e.g., a wetting agent, a sweetener, an aromatic, a preservative, may be contained. Formulations for parenteral administration include a sterilized aqueous solution, a non-aqueous solution, a suspension, an emulsion, a lyophilized formulation and a suppository. Propylene glycol, polyethylene glycol, a plant oil such as olive oil, an injectable ester such as ethyl oleate, etc. may be used as a solvent for the non-aqueous solution or the suspension. Witepsol, macrogol, tween 61, cocoa butter, laurin butter, glycerogelatin, etc. may be used as a base of the suppository.

In an aspect of the present disclosure, the composition may be administered pharmaceutically in the form of a pharmaceutically acceptable salt and may be used alone or in combination with another pharmaceutically active compound. The salt is not specially limited as long as it is a pharmaceutically acceptable one. For example, hydrochloride, sulfate, nitrate, phosphate, hydrofluoride, hydrobromide, formate, acetate, tartrate, lactate, citrate, fumarate, maleate, succinate, methanesulfonate, benzenesulfonate, toluenesulfonate, naphthalenesulfonate, etc. may be used.

In an aspect of the present disclosure, the composition may be administered parenterally or orally depending on purposes and a daily dosage of 0.1-500 mg, specifically 1-100 mg, per 1 kg of body weight may be administered once or several times a day. An administration dosage for a specific patient may vary depending on the body weight, age, sex, health condition and diet of the patient, administration time, administration method, excretion rate, severity of disease, etc.

The pharmaceutical composition according to an aspect of the present disclosure may be prepared into any pharmaceutically appropriate formulations including an oral formulation such as a pill, a granule, a tablet, a soft or hard capsule, a suspension, an emulsion, a syrup, an aerosol, etc. a formulation for external application to skin such as an ointment, a cream, etc., a suppository, an injection, a sterile solution for injection, or the like according to commonly employed methods. Specifically, it may be formulated as an injection or a solution for external application to skin.

The composition according to an aspect of the present disclosure may be administered to a mammal such as rat, mouse, livestock, human, etc. through various routes including parenteral and oral routes. Any possible mode of administration may be expected. For example, the composition may be administered orally, transdermally, rectally, intravenously, intramuscularly, subcutaneously, intrauterinarily or intracerebroventricularly.

The composition according to an aspect of the present disclosure may be administered via various routes that can be easily adopted by those skilled in the art. In particular, the pharmaceutical composition according to an aspect of the present disclosure may be a formulation for external application to skin and may be administered by being applied on the skin surface.

The cell-penetrating peptide according to an aspect of the present disclosure may be an AP-GRR peptide having a sequence of Gly (Arg)ₙ Gly Tyr Lys Cys (1≦n≦20).

In the AP-GRR peptide according to an aspect of the present disclosure, the n may range from 3 to 9.

In an aspect of the present disclosure, the AP-GRR peptide may have a sequence of SEQ ID NO 1.

In an aspect of the present disclosure, when the drug delivery carrier contains a lipid structure, the drug delivery carrier may further contain a stabilizer.

In an aspect of the present disclosure, the stabilizer may be a cholesterol derivative. Specifically, the stabilizer may be cholesterol. The cholesterol derivative refers to a derivative having cholesterol as a backbone.

Specifically, in an aspect of the present disclosure, the drug delivery carrier may contain: a lipid structure covalently bonded to a cell-penetrating peptide or a peptide chain containing the same; a stabilizer; and a physiologically active ingredient.

In an aspect of the present disclosure, the drug delivery carrier may contain the lipid structure and the stabilizer at a molar ratio of 1-3:1-2. Specifically, the molar ratio may be 1.5-3.0:1.0-2.0. More specifically, the lipid structure may be a combination of two or more lipids, and the lipids may be contained at a molar ratio of 1-2:1-2.

In an aspect of the present disclosure, the lipid structure may contain one or more of dioleyl phosphatidylethanolamine, phosphatidylcholine and a distearoyl phosphatidylethanolamine-polyethylene glycol-maleimide (DSPE-PEG-Mal) composite as the lipid structure.

In an aspect of the present disclosure, the drug delivery carrier may contain dioleyl phosphatidylethanolamine, phosphatidylcholine, cholesterol and a distearoyl phosphatidylethanolamine-polyethylene glycol-maleimide (DSPE-PEG-Mal) composite at a molar ratio of 1.0-2.0:1.0-2.0:1.0-3.0:0.01-1.0. Specifically, the molar ratio may be 1.0-1.5:1.0-1.5:1.5-2.5:0.1-0.3. When the lipids are contained at the above molar ratio, the drug delivery carrier may exhibit the most excellent effect of delivering a water-soluble macromolecule into cells.

In an aspect of the present disclosure, the AP-GRR peptide or the peptide chain containing the same covalently bonded to the lipid structure or the polymer particle of the drug delivery carrier may be introduced in an amount of 1 mol % or more, 2 mol % or more, 4 mol % or more, 6 mol % or more, 8 mol % or more or 10 mol % or more, or 15 mol % or less, 10 mol % or less, 8 mol % or less, 6 mol % or less, 4 mol % or less, 2 mol % or less, 1 mol % or less or 0.1 mol % or less, based on the lipid structure or the polymer particle.

In an aspect of the present disclosure, the lipid structure may be a liposome or an emulsion.

In an aspect of the present disclosure, a lipid component of the liposome or the emulsion may be a phospholipid or a nitrolipid having a C₁₂-C₂₄ fatty acid chain.

In an aspect of the present disclosure, the phospholipid may be one or more selected from a group consisting of a natural phospholipid such as egg yolk lecithin (phosphatidylcholine), soy lecithin, lysolecithin, sphingomyelin, phosphatidic acid, phosphatidylserine, phosphatidylglycerol, phosphatidylinositol, phosphatidylethanolamine, diphosphatidylglycerol, cardiolipin and plasmalogen, a hydrogenation product obtainable from the natural phospholipid by a common method, a synthetic phospholipid such as dicetyl phosphate, distearoylphosphatidylcholine, distearoylphosphatidylethanolamine (DSPE), dioleoylphosphatidylethanolamine, dipalmitoylphosphatidylcholine, dipalmitoylphosphatidylethanolamine, dipalmitoylphosphatidylserine, eleostearoylphosphatidylcholine, eleostearoylphosphatidylethanolamine and eleostearoylphosphatidylserine, and a fatty acid mixture obtainable from hydrolysis thereof.

In an aspect of the present disclosure, the phospholipid may be a combination of phosphatidylcholine and phosphatidylethanolamine, a combination of phosphatidylcholine and phosphatidylglycerol, a combination of phosphatidylcholine and phosphatidylinositol, a combination of phosphatidylcholine and phosphatidic acid, a combination of phosphatidylcholine and dioleoylphosphatidylethanolamine or a combination of phosphatidylcholine, dioleoylphosphatidylethanolamine and phosphatidylserine.

Specifically, in an aspect of the present disclosure, the liposome may be a combination of phosphatidylcholine and dioleoylphosphatidylethanolamine.

In an aspect of the present disclosure, a mixing ratio of a maximally contained component to a minimally contained component is 1:5 or smaller.

In an aspect of the present disclosure, the combination may be a combination of phosphatidylcholine, dioleoylphosphatidylethanolamine and phosphatidylserine, and the mixing ratio of the phosphatidylcholine, the dioleoylphosphatidylethanolamine and the phosphatidylserine may be 1-4:1-2:1-2.

In an aspect of the present disclosure, the lipid component may be contained in an amount of 0.001-20 wt % based on the total weight of the liposome suspension or emulsion.

In an aspect of the present disclosure, the polymer particle may comprise one which is biocompatible without inducing inflammation or immune response and is degraded in vivo and its degradation product is also unharmful in vivo.

In an aspect of the present disclosure, the polymer particle may comprise an amphiphilic polymer or a biodegradable aliphatic polyester-based polymer.

In an aspect of the present disclosure, the polymer particle may comprise a biodegradable aliphatic polyester-based polymer based on lactic acid and glycolic acid.

In an aspect of the present disclosure, the biodegradable aliphatic polyester-based polymer may be one or more selected from a group consisting of poly(D,L-lactic acid), poly(L-lactic acid) or poly(D-lactic acid) of Chemical Formula 3, poly(D,L-lactic acid-co-glycolic acid), poly(D-lactic acid-co-glycolic acid) or poly(L-lactic acid-co-glycolic acid) of Chemical Formula 4, poly(caprolactone), poly(valerolactone), poly(hydroxybutyrate), poly(hydroxyvalerate), poly(1,4-dioxan-2-one), poly(orthoester) and a copolymer prepared from monomers thereof:

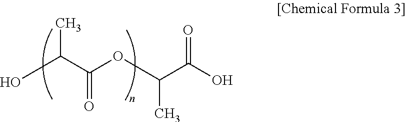

wherein n is an integer 2 or greater

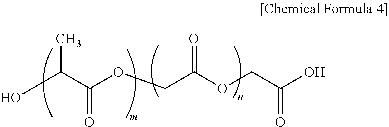

wherein each of m and n are, which may be identical or different, is an integer 2 or greater.

In an aspect of the present disclosure, the biodegradable aliphatic polyester-based polymer may have a molecular weight of 500-100,000 Da on average.

In an aspect of the present disclosure, the AP-GRR peptide (A) and the biodegradable aliphatic polyester-based polymer (B) may be covalently bonded in the form of A-B or A-B-A.

In an aspect of the present disclosure, the covalent bonding may be formed by adding a base, a linker or a multiligand compound between the AP-GRR peptide or the peptide chain containing the AP-GRR peptide and the lipid structure or the polymer particle.

In an aspect of the present disclosure, the drug delivery carrier may have an average particle diameter of 1,000 nm or smaller. In an aspect of the present disclosure, the drug delivery carrier may have an average particle diameter of 100 nm or larger, 200 nm or larger, 300 nm or larger, 400 nm or larger, 500 nm or larger, 600 nm or larger, 700 nm or larger, 800 nm or larger, 900 nm or larger, 1,000 nm or larger or 2,000 nm or larger, or 3,000 nm or smaller, 2,000 nm or smaller, 1,000 nm or smaller, 900 nm or smaller, 800 nm or smaller, 700 nm or smaller, 600 nm or smaller, 500 nm or smaller, 400 nm or smaller, 300 nm or smaller, 200 nm or smaller or 100 nm or smaller.

In an aspect, the present disclosure provides a pharmaceutical composition containing the drug delivery carrier and 0.01-30 wt % of a physiologically active ingredient based on the total weight of the lipid structure or the polymer particle.

In an aspect of the present disclosure, the amount of the physiologically active ingredient encapsulated in the drug delivery carrier may be 0.01-30 wt % based on the total weight of the lipid structure or the polymer particle. Specifically, in an aspect of the present disclosure, the amount of the physiologically active ingredient may be 0.01 wt % or more, 0.05 wt % or more, 0.1 wt % or more, 0.5 wt % or more, 1 wt % or more, 2 wt % or more, 3 wt % or more, 4 wt % or more, 5 wt % or more, 6 wt % or more, 10 wt % or more, 15 wt % or more, 20 wt % or more, 25 wt % or more or 30 wt % or more, or 30 wt % or less, 25 wt % or less, 20 wt % or less, 15 wt % or less, 10 wt % or less, 6 wt % or less, 5 wt % or less, 4 wt % or less, 3 wt % or less, 2 wt % or less, 1 wt % or less, 0.5 wt % or less, 0.1 wt % or less, 0.05 wt % or less or 0.01 wt % or less, based on the total weight of the lipid structure or the polymer particle.

In an aspect of the present disclosure, the composition may be in the form of a formulation selected from a formulation external application to skin, a formulation for oral administration and an injection.

The present disclosure is directed to modifying the surface of a delivery carrier such as a liposome, a polymer nanoparticle, a phospholipid-polymer composite, an emulsion, etc., which has a structure for encapsulating a water-insoluble or water-soluble macromolecule such as a drug, a gene, an oligopeptide, a protein, etc., with an arginine-rich peptide having a glycine (Gly) amino acid residue and a glycine (Gly)-tyrosine (Tyr)-lysine (Lys)-cysteine (Cys) amino acid residue at each end as a newly designed GRR peptide having excellent membrane permeability, in order to increase bioavailability of the delivered substance when it is delivered via various routes including transdermal, oral or injection routes. The AP-GRR peptide has a sequence of Gly (Arg)ₙ Gly Tyr Lys Cys wherein the number of Arg is from 1 to 20, specifically from 3 to 9. In this case, high delivery efficiency can be achieved and the drug delivery carrier can be prepared easily.

In an aspect of the present disclosure, the AP-GRR peptide or the peptide chain containing the AP-GRR peptide may be synthesized, for example, by solid-phase peptide synthesis (SPPS) using an amide 4-methylbenzhydrylamine hydrochloride (MBNA) resin and the ABI 433 synthesizer according to the Fmoc (N-(9-fluorenyl)methoxycarbonyl)/t-butyl method (M. Bodansky, A. Bodansky, The Practice of Peptide Synthesis; Springer: Berlin, Heidelberg, 1984, J. M. Stewart, J. D. Young, Solid Phase Peptide Synthesis, 2^{nd} ed; Pierce Chemical Co: Rockford. Ill., 1984), although not being particularly limited thereto.

In an aspect of the present disclosure, a structure such as a liposome, an emulsion, a polymer particle, etc. may be used in the drug delivery carrier. In an aspect of the present disclosure, when preparing the liposome or the emulsion, a phospholipid or a nitrolipid having a C₁₂-C₂₄ fatty acid chain may be used as a lipid component of the lipid structure. It is useful to be used as a component of a-based drug delivery carrier that can be used in a pharmaceutical composition such as a formulation for external application to skin, a formulation for oral administration, an injection, etc.

Specifically, in an aspect of the present disclosure, the lipid component of the lipid structure may be a phospholipid. Specifically, egg yolk lecithin (phosphatidylcholine), soy lecithin, lysolecithin, sphingomyelin, phosphatidic acid, phosphatidylserine, phosphatidylglycerol, phosphatidylinositol, phosphatidylethanolamine, diphosphatidylglycerol, cardiolipin and plasmalogen, a hydrogenation product obtainable from the natural phospholipid by a common method, a synthetic phospholipid such as dicetyl phosphate, distearoylphosphatidylcholine, distearoylphosphatidylethanolamine (DSPE), dioleoylphosphatidylethanolamine, dipalmitoylphosphatidylcholine, dipalmitoyl phosphatidylethanolamine, dipalmitoylphosphatidylserine, eleostearoylphosphatidylcholine, eleostearoylphosphatidylethanolamine and eleostearoylphosphatidylserine, and a fatty acid mixture obtainable from hydrolysis thereof may be used.

In an aspect of the present disclosure, the lipid may be used either alone or in combination. Specifically, when two or more phospholipids are used in combination, a combination of phosphatidylcholine and phosphatidylethanolamine, a combination of phosphatidylcholine and phosphatidylglycerol, a combination of phosphatidylcholine and phosphatidylinositol, a combination of phosphatidylcholine and phosphatidic acid, a combination of phosphatidylcholine and dioleoylphosphatidylethanolamine, etc. may be used. A mixing ratio of the components may be different depending on their composition. Specifically, a mixing ratio of a maximally contained component to a minimally contained component may be 1:5 or smaller. In this case, it is easy to prepare the lipid-based drug delivery carrier by mixing the two or more phospholipids. For example, when a combination of phosphatidylcholine and dioleoylphosphatidylethanolamine is used, they may be mixed at various molars ratio of 1:1, 2:1, 3:1, 4:1, 5:1, 1:5, 1:4, 1:3, 1:2, etc. And, when three phospholipids are used in combination, for example, when a combination of phosphatidylcholine, dioleoylphosphatidylethanolamine and phosphatidylserine is used, they may be mixed at various molars ratio of 1:1:1, 2:1:1, 3:1:2, 3:2:1. 3:2:2. 4:1:1, 4:2:1, etc.

In an aspect of the present disclosure, the lipid component of the drug delivery carrier is used in an amount of 0.001-20 wt %, specifically 0.2-10 wt %, based on the total weight of the liposome suspension or emulsion. In this case, is easy to prepare the drug delivery carrier.

The polymer particle according to an aspect of the present disclosure should be biocompatible without inducing inflammation, immune response, etc. should be degraded in vivo. And its degradation product should also be unharmful in vivo. As a polymer satisfying these requirements, a biodegradable aliphatic polyester-based polymer having polymer lactic acid and glycolic acid as basic units, which has been approved by the US Food and Drug Administration (FDA) is used the most widely. Representative examples include poly(D,L-lactic acid), poly(L-lactic acid) or poly(D-lactic acid) of Chemical Formula 3, poly(D,L-lactic acid-co-glycolic acid), poly(D-lactic acid-co-glycolic acid) or poly(L-lactic acid-co-glycolic acid) of Chemical Formula 4, poly(caprolactone), poly(valerolactone), poly(hydroxybutyrate), poly(hydroxyvalerate), poly(1,4-dioxan-2-one), poly(orthoester) and a copolymer prepared from monomers thereof:

In an aspect of the present disclosure, the molecular weight of the biodegradable aliphatic polyester-based polymer is not particularly limited. However, since the structural instability of the drug delivery carrier may increase if the molecular weight is smaller than 500 Da or greater than 100,000 Da, it may have a weight-average molecular weight of 500-100,000 Da, specifically 5,000-50,000 Da.

In an aspect of the present disclosure, the molecular weight of the biodegradable aliphatic polyester-based polymer may be 1,000 Da or greater, 2,000 Da or greater, 3,000 Da or greater, 4,000 Da or greater, 5,000 Da or greater, 6,000 Da or greater, 7,000 Da or greater, 8,000 Da or greater, 9,000 Da or greater, 10,000 Da or greater, 11,000 Da or greater, 12,000 Da or greater, 13,000 Da or greater, 14,000 Da or greater, 15,000 Da or greater, 20,000 Da or greater, 30,000 Da or greater, 50,000 Da or greater or 100,000 Da or greater, or 200,000 Da or smaller, 150,000 Da or smaller, 100,000 Da or smaller, 90,000 Da or smaller, 80,000 Da or smaller, 70,000 Da or smaller, 60,000 Da or smaller, 50,000 Da or smaller, 40,000 Da or smaller, 30,000 Da or smaller, 20,000 Da or smaller, 10,000 Da or smaller, 5,000 Da or smaller, 3,000 Da or smaller or 1,000 Da or smaller, although not being limited thereto.

In an aspect of the present disclosure, for the poly(D,L-lactic acid-co-glycolic acid) of Chemical Formula 4, biodegradable polymers having various degradation rates may be obtained by controlling the ratio of the lactic acid and glycolic acid monomers or by modifying the polymer synthesis process. Such biodegradable aliphatic polyester-based polymers have long been used as drug delivery carriers or surgical sutures with proven biocompatibility.

In an aspect of the present disclosure, the AP-GRR peptide (A) and the biodegradable aliphatic polyester-based polymer (B) may be covalently bonded in the form of A-B or A-B-A, although not being particularly limited thereto. This may be achieved by replacing a carboxyl group and a hydroxyl group present on each end of the biodegradable aliphatic polyester-based polymer with other functional groups favorable for covalent bonding and reacting the terminal functional groups with a terminal group of the AP-GRR peptide or a peptide chain containing the AP-GRR peptide. For example, a polymer wherein the AP-GRR peptide or a peptide chain containing the AP-GRR peptide is covalently bonded to poly(D,L-lactic acid-co-glycolic acid) may be synthesized by covalently bonding the terminal functional group of maleimide-substituted poly(D,L-lactic acid-co-glycolic acid) with a thiol-substituted AP-GRR peptide.

In an aspect of the present disclosure, the covalent bonding may be formed by adding a base, a linker or a multiligand compound between the AP-GRR peptide or the peptide chain containing the AP-GRR peptide and the lipid structure or the polymer particle, although not being particularly limited thereto.

In an aspect of the present disclosure, the physiologically active ingredient encapsulated inside the drug delivery carrier may be water-soluble or water-insoluble and is not limited as long as it can be applied in vivo. For example, it may be an extract derived from an animal, a plant or a microorganism and may be either a single ingredient or a mixture of two or more ingredients depending on purposes. An ingredient effective in improving skin whitening, preventing wrinkling and aging, treating a disease, or the like may be used. The physiologically active ingredient may be contained in an amount of 0.01-30 wt %, specifically 0.1-20 wt %, based on the total weight of the lipid structure or the polymer particle. In this case, the composition may be easily prepared as a formulation for external application to skin, a formulation for oral administration, an injection, etc.

It is desired that the drug delivery carrier prepared using the AP-GRR peptide according to an aspect of the present disclosure has an average particle diameter as small as possible. When considering colloidal stability, it is desired that the average particle diameter is 1,000 nm or smaller, specifically 500 nm or smaller.

A method for preparing the drug delivery carrier according to an aspect of the present disclosure is not particularly limited. For example, it may be prepared as follows.

As a method for forming the drug delivery carrier wherein the physiologically active ingredient is encapsulated using the lipid component presented in the present disclosure, a method of dissolving a phospholipid and a stabilizer in an organic solvent, evaporating the solvent, forming a lipid film by reducing pressure, adding an aqueous solution and then applying ultrasonic waves, a method of dispersing a phospholipid and a stabilizer dissolved in an organic solvent in an aqueous solution and then applying ultrasonic waves, a method of dispersing or dissolving a phospholipid and a stabilizer in an organic solvent and then extracting or evaporating the organic solvent with excess water, a method of dispersing or dissolving a phospholipid and a stabilizer in an organic solvent, stirring vigorously using a homogenizer or a high-pressure emulsifier and then evaporating the solvent, a method of dispersing or dissolving a phospholipid and a stabilizer in an organic solvent and then dialyzing with excess water, a method of dispersing or dissolving a phospholipid and a stabilizer in an organic solvent and then slowly adding water, or the like may be used, although not being limited thereto.

In the above-described methods, the phospholipid and the stabilizer in the organic solvent may be dissolved by applying mechanical force or by heating to 20-100° C., specifically to 70° C. or lower.

When the physiologically active ingredient is water-soluble, the physiologically active ingredient is dissolved in water or an aqueous solution and added in the step where the aqueous solution or water is added. When the physiologically active ingredient is water-insoluble, the physiologically active ingredient may be dissolved in an organic solvent and then added to the organic solvent phase where the lipid component is present.

As the organic solvent used to dissolve the phospholipid and the stabilizer or the water-insoluble physiologically active ingredient, one or more solvent selected from acetone, dimethyl sulfoxide, dimethylformamide, N-methylpyrrolidone, dioxane, tetrahydrofuran, acetic acid, ethyl acetate, acetonitrile, methyl ethyl ketone, methylene chloride, chloroform, methanol, ethanol, ethyl ether, diethyl ether, hexane and petroleum ether may be used, although not being limited thereto.

As a method for forming the polymer particle according to the present disclosure, a method of dispersing a polymer directly in an aqueous solution and then applying ultrasonic waves, a method of dispersing or dissolving a polymer in an organic solvent and then extracting or evaporating the organic solvent with excess water, a method of dispersing or dissolving a polymer in an organic solvent, stirring vigorously using a homogenizer or a high-pressure emulsifier and then evaporating the solvent, a method of dispersing or dissolving a polymer in an organic solvent and then dialyzing with excess water, a method of dispersing or dissolving a polymer in an organic solvent and then slowly adding water, a method of using a supercritical fluid, or the like may be used (T. Niwa et al., J. Pharm. Sci (1994) 83, 5, 727-732; C. S. Cho et al., Biomaterials (1997) 18, 323-326; T. Govender et al., J. Control. Rel. (1999) 57, 171-185; M. F. Zambaux et al., J. Control. Rel. (1998) 50, 31-40).

The organic solvent that can be used to prepare the polymer particle of the present disclosure includes acetone, dimethyl sulfoxide, dimethylformamide, N-methylpyrrolidone, dioxane, tetrahydrofuran, ethyl acetate, acetonitrile, methyl ethyl ketone, methylene chloride, chloroform, methanol, ethanol, ethyl ether, diethyl ether, hexane or petroleum ether, although not being limited thereto. The solvent may be used either alone or in combination.

The distearoyl phosphatidylethanolamine-polyethylene glycol-maleimide (DSPE-PEG-Mal) composite used in the present disclosure may have a structure of Chemical Formula 5.

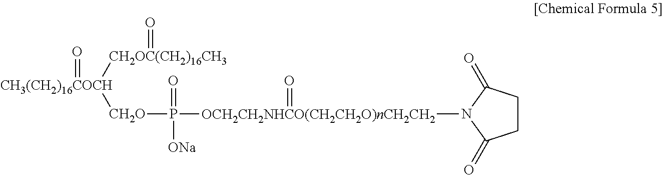

Hereinafter, the present disclosure will be described in detail through examples, comparative examples and test examples. The materials, reagents, operations, etc. described in the following examples can be changed appropriately within the scope of the present disclosure. Accordingly, the scope of the present disclosure is not limited by the examples.

### EXAMPLES AND COMPARATIVE EXAMPLES

### Preparation of Drug Delivery Carrier

A peptide chain containing an AP-GRR peptide was synthesized by solid-phase peptide synthesis (SPPS) using an amide 4-methylbenzhydrylamine hydrochloride (MBNA) resin and the ABI 433 synthesizer according to the Fmoc (N-(9-fluorenyl)methoxycarbonyl)/t-butyl method and purified by reversed-phase high-performance liquid chromatography to a purity of 90% or higher. A successful synthesis was confirmed by measuring molecular weight using a mass analyzer (Agilent 1100 series). It was confirmed that an AP-GRR peptide having an amino acid sequence of SEQ ID NO 1 was synthesized. A lipid structure wherein the AP-GRR peptide was introduced to the surface thereof was prepared as follows.

After mixing the lipids described in Table 1 at the specified lipid molar ratios and dissolving in a mixture organic solvent of chloroform and methanol (95:5, v/v), a film was formed by evaporating the solvent. For example, in Example 2, after mixing dioleyl phosphatidylethanolamine, phosphatidylcholine, cholesterol and a distearoyl phosphatidylethanolamine-polyethylene glycol-maleimide composite at a molar ratio of 1.5:1.1:2:0.4 and dissolving in a mixture organic solvent of chloroform and methanol (95:5, v/v), a film was formed by evaporating the solvent.

After adding the fluorophore rhodamine B (Sigma-Aldrich, CAS NO. 81-88-9) or dextran-RITC (number-average molecular weight=˜10,000 Da) (Sigma-Aldrich) dissolved in PBS (Welgene) to the film having the organic solvent removed, a liposome lipid dispersion was prepared by applying ultrasonic waves. After adding the AP-GRR peptide dissolved in PBS to the prepared liposome lipid dispersion, reaction was conducted by stirring at room temperature (˜25° C.). Through the reaction, an AP-GRR-introduced liposome was prepared from bonding between the thiol group of the AP-GRR peptide and the maleimide functional group protruding on the liposome surface. This bonding is known to be very stable. The concentration of the lipid in the final aqueous solution was 0.2 wt %.

Examples and comparative examples were prepared by the above-described method with the lipid compositions and lipid molar ratios described in Table 1. Examples 1-5 are AP-GRR-introduced liposomes, but a fluorophore was not attached in Example 1. Comparative Example 1 is a non-AP-GRR-introduced liposome with no fluorophore attached, Comparative Examples 2 and 4 are fluorophores dissolved in PBS, and Comparative Examples 3 and 5 are non-AP-GRR-introduced liposomes having a fluorophore attached. The lipid molar ratios of Examples 1-5 were calculated based on the molecular weight of DSPE, by subtracting the molecular weights of PEG and Mal from that of DSPE-PEG-Mal. In Example 6, an amphiphilic polymer was further added to the composition of Example 5 in order to investigate the effect of introduction of the amphiphilic polymer. The poly(hydroxyethyl methacrylate-co-stearyl methacrylate) copolymer used in Example 6 has a number-average molecular weight of 47,552 (PDI=2.20).

The amphiphilic polymer was polymerized by adding to 150 g of ethanol a hydroxyethyl methacrylate monomer (purchased from Sigma-Aldrich) and a stearyl methacrylate monomer purchased from Sigma-Aldrich) at a molar ratio of 0.0995654:0.232315 and performing polymerization by adding 0.003319 mol of azobisisobutyronitrile (AIBN, purchased from Junsei) as a radical polymerization initiator and stirring overnight at 75° C. After the polymerization, heating was stopped and the mixture was allowed to cool to room temperature. Then, after stirring the mixture while slowing adding 5-10 times of ether based on the ethanol solution, the solvent was removed by filtering and the resulting precipitate was recovered. The obtained precipitate was vacuum-dried to obtain 40 g of a poly(hydroxyethyl methacrylate-co-stearyl methacrylate) copolymer powder.

Example 6 was prepared in the same manner except that the amphiphilic polymer was added together when the lipid was dissolved in the organic solvent.

TABLE 1

| | | | | | Concentration |
|---|---|---|---|---|---|
| | | Lipid molar | Amphiphilic | Introduction | of fluorophore |
| | Lipid composition | ratio | polymer | of AP-GRR | in final solution |
| Example 1 | DOPE:PC:Chol:DSPE- | 1.5:1.1:2:0.4 | X | ◯ | — |
| | PEG-Mal | | | | |
| Example 2 | DOPE:PC:Chol:DSPE- | 1.5:1.1:2:0.4 | X | ◯ | Rhodamine B, |
| | PEG-Mal | | | | 0.05 wt % |
| Example 3 | DOPE:PC:Chol:DSPE- | 1.5:1.3:2:0.2 | X | ◯ | Rhodamine B, |
| | PEG-Mal | | | | 0.05 wt % |
| Example 4 | DOPE:PC:Chol:DSPE- | 1.5:1.1:2:0.4 | X | ◯ | Dextran-RITC, |
| | PEG-Mal | | | | 1 wt % |
| Example 5 | DOPE:PC:Chol:DSPE- | 1.5:1.3:2:0.2 | X | ◯ | Dextran-RITC, |
| | PEG-Mal | | | | 1 wt % |
| Example 6 | DOPE:PC:Chol:DSPE- | 1.5:1.3:2:0.2 | Poly(hydroxyethyl | ◯ | Dextran-RITC, |
| | PEG-Mal | | methacrylate-co- | | 1 wt % |
| | | | stearyl | | |
| | | | methacrylate) | | |
| | | | copolymer (0.06 | | |
| | | | wt % in final | | |
| | | | solution) | | |
| Comparative | DOPE:PC:Chol | 1.5:1.5:2 | | X | — |
| Example 1 | | | | | |
| Comparative | — | — | | — | Rhodamine B, |
| Example 2 | | | | | 0.05 wt % |
| Comparative | DOPE:PC:Chol | 1.5:1.5:2 | | X | Rhodamine B, |
| Example 3 | | | | | 0.05 wt % |
| Comparative | — | — | | — | Dextran-RITC, |
| Example 4 | | | | | 1 wt % |
| Comparative | DOPE:PC:Chol | 1.5:1.5:2 | | X | Dextran-RITC, |
| Example 5 | | | | | 1 wt % |

DOPE: phosphatidylethanolamine (Doosan Biotech), PC: phosphatidylcholine (Lipoid), Chol: cholesterol (Sigma-Aldrich), DSPE-PEG-Mal (molecular weight = 2941.605; NOF): distearylphosphatidylethanolamine-polyethylene glycol-maleimide composite (N-[(3-maleimide-1-oxopropyl)aminopropyl polyethylene glycol-carbamyl]distearoylphosphatidylethanolamine)

[Preparation of Mixture with o/w Nanoemulsion]

In order to compare absorption into skin and particle size of the drug delivery carriers with or without the amphiphilic polymer introduced upon mixing with an o/w nanoemulsion, Examples 7 and 8 were prepared as test samples by mixing the drug delivery carriers of Example 5 and Example 6 with an o/w nanoemulsion.

Specifically, an aqueous phase and an oil phase were prepared as described in Table 2.

TABLE 2

| | Components | Amount (g) |
|---|---|---|
| Oil phase | Stearic acid | 0.8 |
| | Cetyl alcohol | 1.2 |
| | Pentaerythrityl tetraethylhexanoate | 4.0 |
| | Silicone oil | 4.0 |
| | Hydrogenated lecithin | 1.5 |
| | Inulin lauryl carbamate | 1.0 |
| Aqueous | Purified water (D.I. water) | 72.0 |
| phase | Tetraethanolamine | 0.1 |
| | Phenoxyethanol | 0.3 |
| | Glycerin | 5.0 |
| | Polyethylene glycol (number-average | 2.0 |
| | molecular weight = 4,000) | |
| | Butylene glycol | 8.0 |
| | Poly(methacrylic acid) copolymer (ETD2020) | 0.1 |

The aqueous phase and the oil phase of the above compositions were separately heated to 70° C. Then, emulsification (homogeneous mixing) was conducted at 7,000 rpm for 3 minutes using a homomixer (T.K. Homomixer Mark II, Takushu Kika Kogyo Ltd., Japan) while slowly adding the oil phase to the aqueous phase. The obtained o/w emulsion was further treated with a high-pressure emulsifier (Microfluidics Corp., USA) at 1000 bar for 3 cycles to obtain a nanoemulsion with an average particle size of about 150 nm. The prepared nanoemulsion was mixed with each of the nanoemulsions of Example 5 and Example 6 at a weight ratio of 1:1 under agitation. The obtained mixture compositions of the drug delivery carriers of Example 5 and Example 6 and the nanoemulsion were designated as Example 7 and Example 8.

### Test Example 1

### Analysis of Particle Size and Measurement of Surface Potential of Drug Delivery Carrier

For the liposome solutions of Examples 1-8 and Comparative Examples 1, 3 and 5, particle size analysis and surface potential measurement were conducted using the Malvern Zetasizer. The result is shown in Table 3.

TABLE 3

| | Average particle size (PDI) | | Surface potential (STD) | |
|---|---|---|---|---|
| Comparative | 81.34 nm | (0.255) | −27.5 mV | (5.16) |
| Example 1 | | | | |
| Example 1 | 108.3 nm | (0.211) | 34.8 mV | (6.34) |
| Comparative | 117.7 nm | (0.292) | −19.9 mV | (6.58) |
| Example 3 | | | | |
| Example 3 | 122.6 nm | (0.210) | 14.7 mV | (9.7) |
| Example 2 | 113.2 nm | (0.210) | 22.1 mV | (13.5) |
| Comparative | 127.3 nm | (0.266) | −22.2 mV | (3.84) |
| Example 5 | | | | |
| Example 5 | 115.5 nm | (0.376) | 11.8 mV | (8.29) |
| Example 4 | 102.1 nm | (0.188) | 20.6 mV | (9.75) |
| Example 6 | 200.6 nm | (0.284) | 18.2 mV | (8.26) |

| | | | |
|---|---|---|---|
| Example 7 | 856 nm | (0.607) | — |
| Example 8 | 192 nm | (0.301) | — |

From Table 3, it can be seen that the samples of the comparative examples and the examples are uniform with an average particle size of around 100 nm.

It can be also seen that the lipid structures with the AP-GRR peptide introduced show the change in the surface potential from negative to positive values as compared to those without the AP-GRR peptide. In addition, it can be seen that Examples 2 and 4 where 8 mol % of AP-GRR was introduced have larger positive surface potential values than Examples 3 and 5 where 4 mol % of AP-GRR was introduced. From these results, it was confirmed that the physical properties of the drug delivery carriers of the present disclosure changed due to the introduction of AP-GRR.

From the particle size analysis result of the nanoemulsions, it can be seen that Example 8 with the amphiphilic polymer introduced has a smaller and shows average particle size and higher particle size stability than Example 7 without the amphiphilic polymer. It is thought that, for Example 7, as new recombination occurs between the liposome containing the cell-permeating peptide and the nanoemulsion, the particle size and the PDI value have increased. This seems to have indirectly resulted from the structural instability in emulsion, which is one of the disadvantages of the liposome formulation. However, for Example 8, the particle size was similar to that of Example 6 prepared from the lipid structure and no new particles with a large particle size were observed. It is thought that the amphiphilic polymer acts as a protective colloid by binding the lipid bilayer of the liposome.

### Test Example 2

### Structural Analysis of Drug Delivery Carrier by TEM

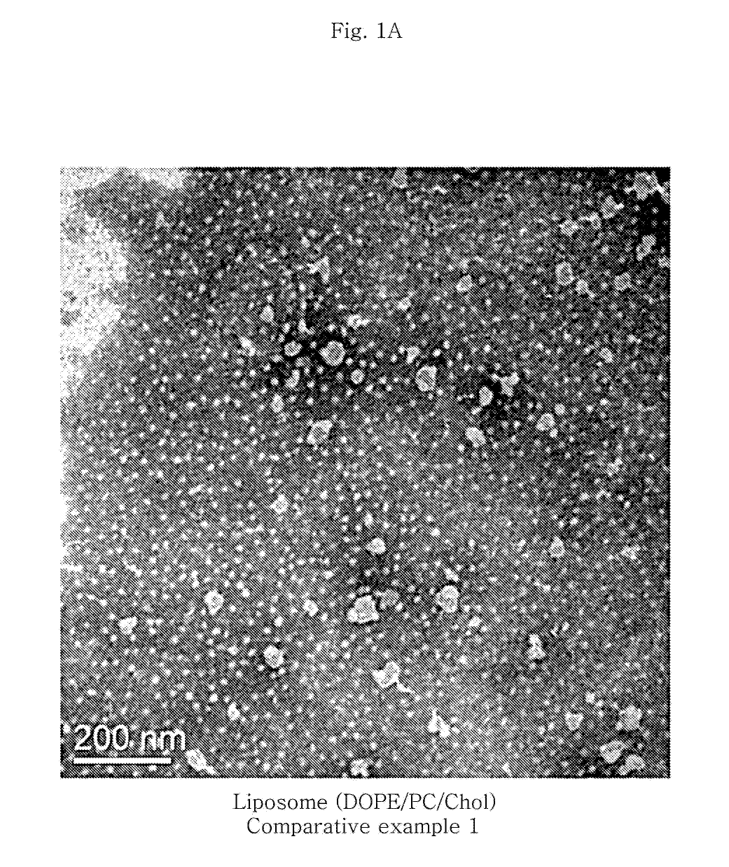

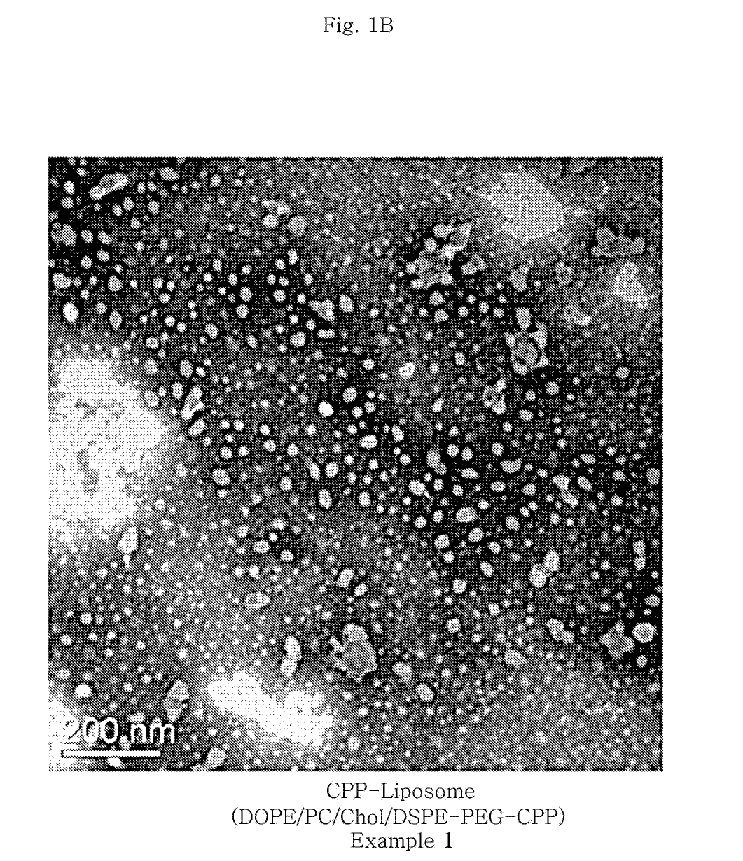

Structural analysis was conducted for Comparative Example 1 and Example 1 using a transmission electron microscope (Libra 120, Carl Zeiss, accelerating voltage=120 kV). The result is shown in FIG. 1.

From FIG. 1, it can be seen that the liposome structure of the drug delivery carrier of Example 1 wherein AP-GRR was introduced to the liposome became slightly larger as compared to Comparative Example 1 wherein AP-GRR was not introduced. Also, it can be seen that Comparative Example 1 and Example 1 show similar liposome structure in spite of the introduction of AP-GRR. As a result, it was confirmed that the drug delivery carrier of the present disclosure which has a structure capable of delivering a macromolecule is structurally similar to the general liposome. Despite the structural similarity to the general liposome, the drug delivery carrier of the present disclosure is capable of easily delivering a water-soluble macromolecule into cells as demonstrated in the following test examples.

### Test Example 3

### Evaluation of Ability of Delivering into Cells of Drug Delivery Carrier Through Flow Cytometry

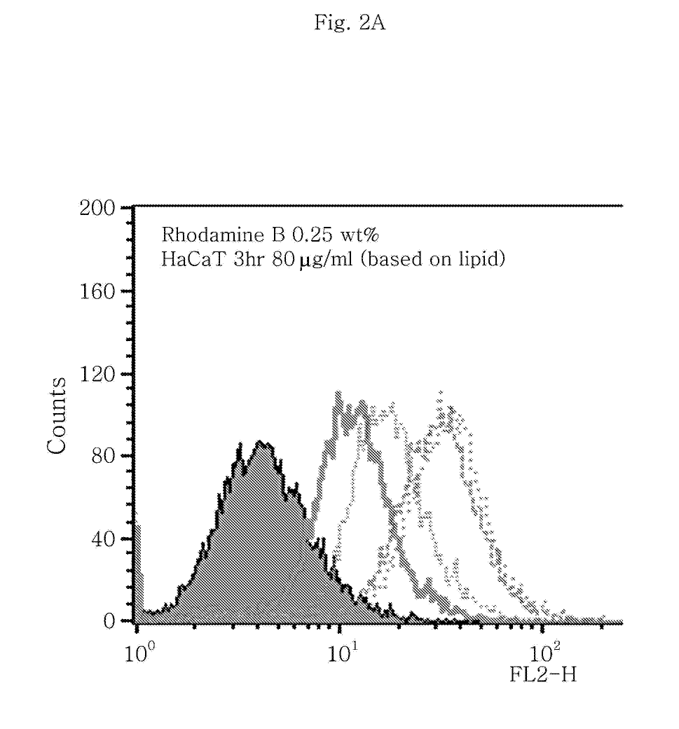

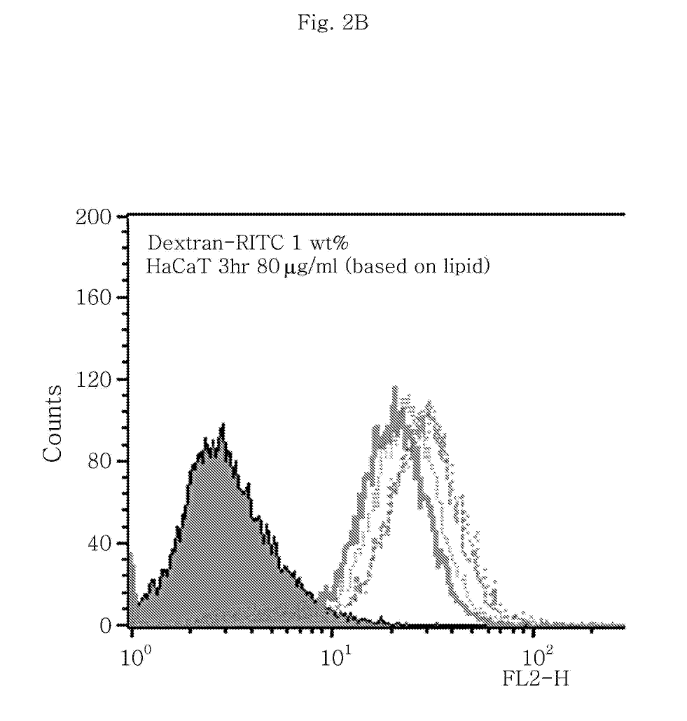

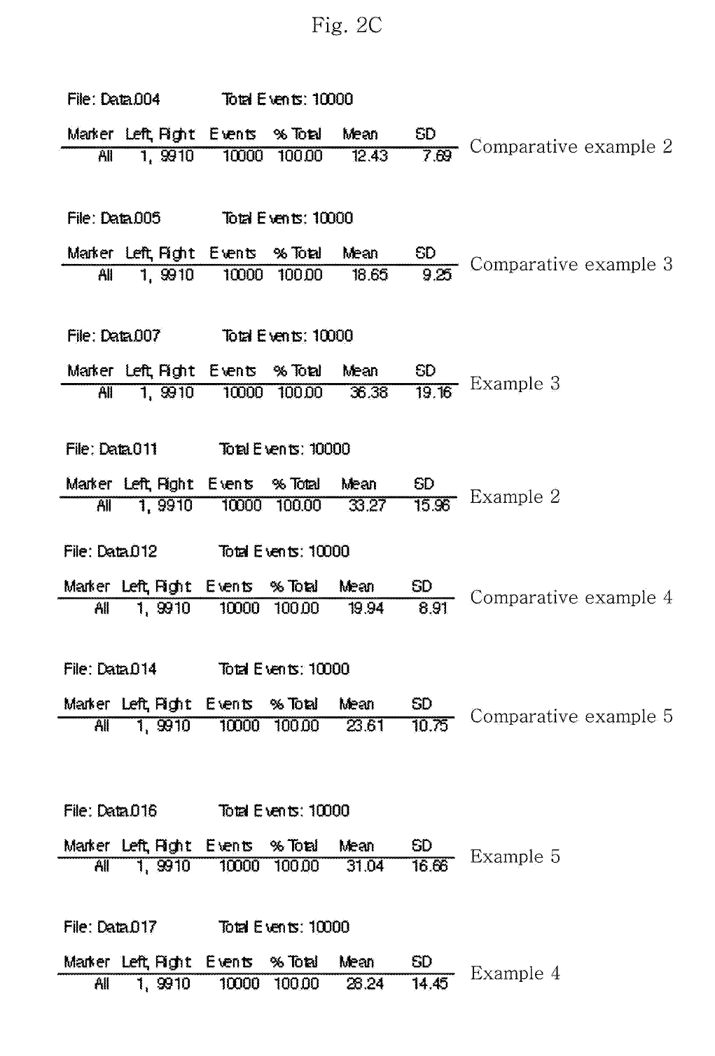

FACS analysis was conducted for Examples 2-5 and Comparative Examples 2-5 in order to evaluate the ability of delivering into cells of the drug delivery carriers. The liposome systems of the examples and the comparative examples were added to HaCaT cells (acquired from Cell Line Service (CLS)) that had been cultured previously. After incubation at 37° C. for 4 hours, the cells were recovered from each sample group and subjected to FACS analysis after dispersing in PBS. Red fluorescence from 10,000 HaCaT cells per each group was measured using the BD FACSCalibur instrument (Beckton Dickinson Bioscience, San Jose, Calif.) and the acquired data were analyzed with the CellQuest software. Through this, the amount of rhodamine B delivered into the cells was compared and analyzed quantitatively. The result is shown in FIG. 2. In FIG. 2, (A) shows the FACS analysis result for rhodamine B of Examples 2 and 3 and Comparative Examples 2 and 3, and (B) shows the result for dextran-RITC of Examples 4 and 5 and Comparative Examples 4 and 5. (C) shows numerical data obtained from the graphs as mean values and standard deviations. In the graphs, the y-axis denotes the number of cells and the x-axis denotes the amount delivered into the cells.

From (A) and (C) in FIG. 2, where the graphs correspond to Example 3, Example 2, Comparative Example 3 and Comparative Example 2 from right to left, it can be seen that the examples wherein AP-GRR was introduced show larger amounts delivered into the cells as compared to the comparative examples wherein AP-GRR was not introduced. Accordingly, it was confirmed that the liposome show better ability of delivering into cells than PBS and that the drug delivery carrier of the present disclosure wherein AP-GRR was introduced to the liposome has better delivering ability than the simple liposome. Through this, it was confirmed that the drug delivery carrier according to the present disclosure can deliver small water-soluble materials such as rhodamine B into the cells well. In addition, when comparing Examples 2 and 3 wherein different amounts of AP-GRR were introduced, it can be seen that Example 2 wherein the introduction amount was 8 mol % based on the lipids constituting the liposome showed a smaller mean value than Example 3 wherein the introduction amount was 4 mol %. Accordingly, an AP-GRR introduction amount of 4 mol % seems suitable although the difference is insignificant.

From (B) and (C) in FIG. 2, where the graphs correspond to Example 5, Example 4, Comparative Example 5 and Comparative Example 4 from right to left, it can be seen that the examples wherein AP-GRR was introduced show larger amounts delivered into the cells as compared to the comparative examples wherein AP-GRR was not introduced. Accordingly, it was confirmed that the liposome show better ability of delivering into cells than PBS and that the drug delivery carrier of the present disclosure wherein AP-GRR was introduced to the liposome has better delivering ability than the simple liposome. Examples 4 and 5 showed similar results as those of Examples 2 and 3 for rhodamine B although the encapsulated dextran-RITC is a polymer with a molecular weight about 20 times that of rhodamine B. Accordingly, it was confirmed that the drug delivery carrier of the present disclosure also exhibits excellent ability of delivering water-soluble macromolecules into cells. In addition, when comparing Examples 4 and 5 wherein different amounts of AP-GRR were introduced, it can be seen that Example 5 wherein the introduction amount was 4 mol % showed a larger mean value as in the experiment for rhodamine B.

### Test Example 4

### Evaluation of Delivery into Cells by Immunofluorescence Staining and Confocal Laser Scanning Microscopy

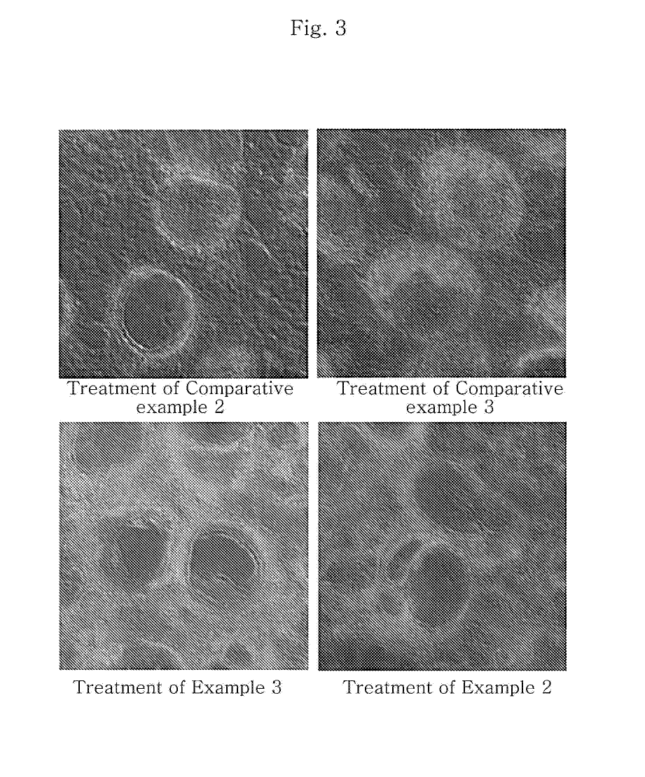

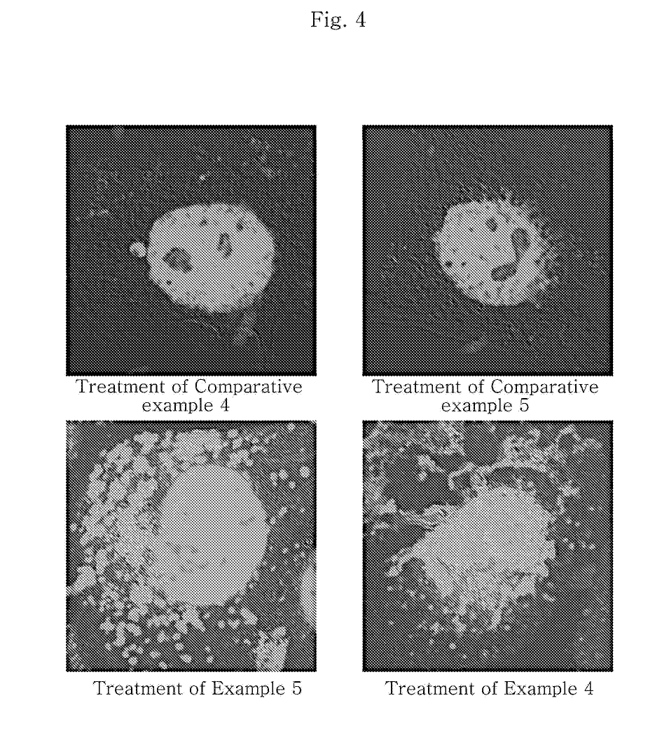

HaCaT cells (acquired from Cell Line Service (CLS)) in DMEM (Lonza) supplemented with 10 wt % FBS (GIBCO) and 100 IU penicillin G (Lonza) were seeded onto an 8-well chamber slide at a density of 25,000 cells/well. After washing the wells with phosphate buffered saline (PBS), the cells were treated for 3 hours with a control medium containing nothing or with the examples or comparative examples diluted in media. The treated cells were subjected to immunofluorescence (IF) staining. After washing each wall using PBS supplemented with 1 mM and CaCl₂ and 1 mM MgCl₂ (the same PBS was used in this test example), the cells were fixed by reacting with 3.5 wt % paraformaldehyde at room temperature for 10 minutes. The fixed cells were washed again three times with PBS for 10 minutes. Then, the cells were treated with 0.1% Triton X-100 for 5 minutes. After washing again three times with PBS for 10 minutes, the cells treated with propidium iodide (PI) for about 3 minutes to stain the nuclei. After washing again three times with PBST (prepared by mixing PBS with 0.05 wt % Tween 20; Tween 20 was purchased from Sigma; PBS used to prepare PBST did not contain calcium chloride or magnesium chloride), a mounting solution was added and a cover glass was placed on the slide. The stained slide was imaged using a confocal laser scanning microscope (Zeiss). The result is shown in FIG. 3 and FIG. 4. FIG. 3 shows the result for the liposomes containing the small water-soluble molecule rhodamine B as a fluorophore, and FIG. 4 shows the result for the liposomes containing the water-soluble macromolecule dextran-RITC as a fluorophore. In the images, the red regions indicate rhodamine B or dextran-RITC, and the blue regions indicate the nuclei.

From FIG. 3, it can be seen that the simple liposome of Comparative Example 3 shows better delivery into cells than the PBS of Comparative Example 2 and that the drug delivery carrier of the present disclosure with AP-GRR introduced exhibits remarkably better delivery into cells than the simple liposome.

From FIG. 4, it can be seen that a result similar to that of rhodamine B is achieved for dextran-RITC whose molecular weight is about 20 times greater. From the images of Comparative Examples 4 and 5, it can be seen that the dextran-RITC was hardly delivered into the cells. In contrast, the images of Examples 4 and 5 show that large amounts of the fluorophores shown in red color were delivered into the cells. Accordingly, it was confirmed that the drug delivery carrier of the present disclosure exhibits a remarkable effect of delivering a water-soluble macromolecule into cells.

### Test Example 5

### Evaluation of Dermal Stability

A patch test of attaching a patch containing the liposome of the examples or comparative examples was conducted for 18 female and 12 male adult subjects (32.5 years on average) in order to investigate the dermal stability of Examples 1-5 and Comparative Examples 1-5. After attaching the patch for 28 hours, first evaluation was made 30 minutes after removal of the patch and second evaluation was made 96 hours later. Skin irritation was evaluated with naked eyes by giving weights depending on the degree of positive skin response. The result is shown in Table 4.

TABLE 4

| Test substances | Average response | Evaluation result |
|---|---|---|
| Examples 1-5 | 0 | Unirritable |
| Comparative | 0 | Unirritable |
| Examples 1-5 | | |

From Table 4, it can be seen that all the examples and the comparative examples do not irritate skin when contained in compositions. Accordingly, it was confirmed that a cosmetic composition containing the liposome of the present disclosure has superior dermal stability.

### Test Example 6

### Evaluation of Qualitative Effect of Drug Delivery Carrier Through Transdermal Absorption Experiment

Transdermal absorption experiment was conducted for Comparative Examples 2-5, Example 3, Example 5, Example 7 and Example 8. For the transdermal absorption experiment, pig ear skins obtained from a slaughterhouse were used. After washing the skin, transdermal absorption experiment of the fluorophores contained in the comparative examples and the examples was conducted for 4 hours and 18 hours, respectively, using a Franz-type vertical diffusion cell system (Microette Plus Auto Sampling System, Hanson Research, USA).

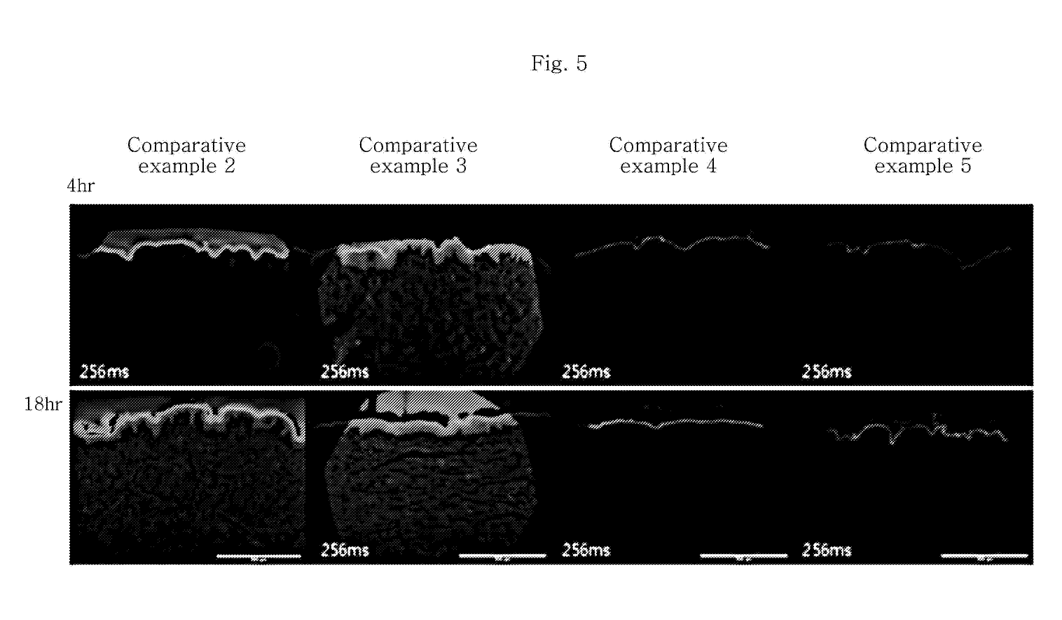

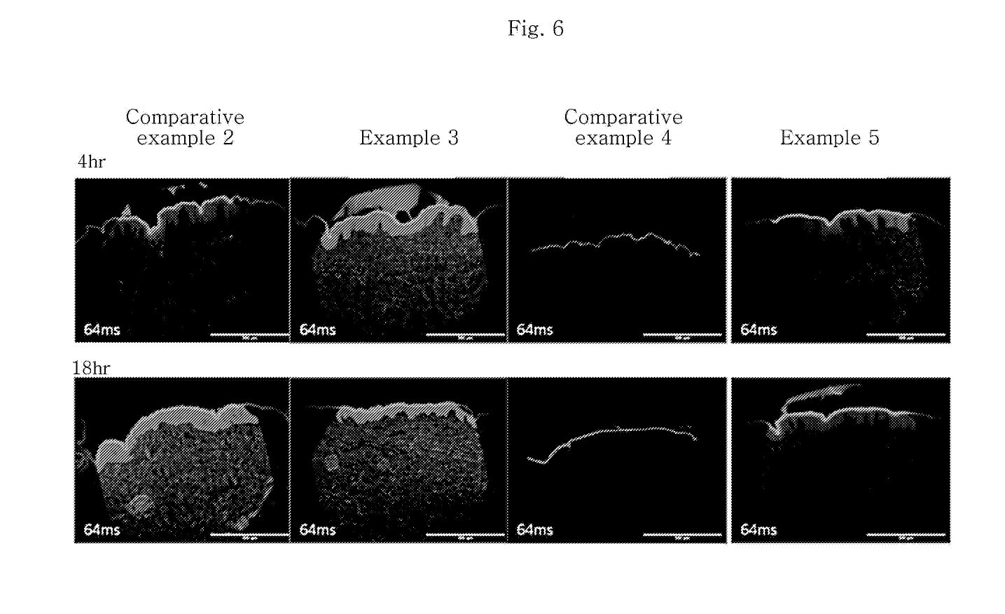

Then, the pig ear skin was put in a mold and was embedded with the OCT compound (#4583, SAKURA Tissue-Tek, USA). Then, the tissue was frozen rapidly at −196° C. using liquid nitrogen. The frozen pig ear skin was sectioned to a thickness of 6 μm using a cryostat (CM1950, Leica, Germany) and attached on a silane-coated slide glass. The slide glass was dried for 10 minutes at room temperature (25° C.) in a shaded place and observed using an optical microscope (BX53, Olympus, Japan). The observation was made under the same fluorescence intensity and exposure time. Representative images taken using a cooled digital color camera (DP72, Olympus, Japan) are shown in FIGS. 5 and 6. In FIGS. 5 and 6, the white scale bar corresponds to 500 μm. In FIG. 5, the exposure time was set to be 256 ms. In FIG. 6, the exposure time was set to be 64 ms. A mercury lamp (U-HGLGPS, Olympus, Japan) was used as a fluorescent light source and the fluorescence intensity was set to 6 from among the selectable values (0, 3, 6, 12, 25, 50 and 100).

From FIG. 5, it can be seen that, for rhodamine B which has a relatively small molecular weight, more than a certain amount is absorbed transdermally when it is dissolved in PBS and absorbed for 18 hours (Comparative Example 2) and is absorbed for 4 hours when the general liposome is used (Comparative Example 3). This suggests that the liposome enhances the absorption of the water-soluble component by disturbing the stratum corneum lipids. But, the dextran-RITC having a large molecular weight was hardly absorbed transdermally (Comparative Examples 4 and 5).

From FIG. 6, it can be seen that rhodamine B is absorbed remarkably in 4 hours when the drug delivery carrier according to an aspect of the present disclosure was used as compared to when it was dissolved in PBS. Further, it can be seen that the rhodamine B encapsulated in the drug delivery carrier of Example 3 shows wider and broader fluorescence as compared to when the general liposome of Comparative Example 3 was used (FIG. 5). The enhanced transdermal absorption is thought to result from the cationic charge of the polyarginine group of the cell-penetrating peptide in addition to the effect of disturbing the stratum corneum lipids of the liposome.

In addition, for the dextran-RITC which was hardly absorbed transdermally for Comparative Examples 4 and 5, transdermal absorption was observed when the drug delivery carrier according to an aspect of the present disclosure was used (Example 5). Accordingly, it can be seen that the drug delivery carrier according to an aspect of the present disclosure exhibits a distinct and remarkable effect of transdermally delivering a polymer material having a large molecular weight of about 10,000 Da.

### Test Example 7

### Evaluation of Quantitative Effect of Drug Delivery Carrier Through Transdermal Absorption Experiment

From the pig ear skin to which each of the comparative examples and examples was absorbed for 4 hours using the Franz cell in Test Example 6, a stratum corneum sample, a skin tissue sample excluding the stratum corneum and a receptor sample were prepared as follows. The stratum corneum sample was prepared by stripping the surface of the skin tissue 3 times with a 3M tape after 6-mm biopsy and extracting the tape using 6 mL of a mixture solvent of water and methanol (1:1). The skin tissue sample excluding the stratum corneum was obtained by extracting the tissue sample remaining after the tape stripping with 2 mL of a mixture solvent of water and methanol (1:1). The receptor sample was obtained by adding the receptor part remaining after the absorption to 1 mL of PBS.

The prepared samples were analyzed using a spectrophotometer (F4500, Hitachi). The analysis condition was as follows.

Analysis condition:
ex slit (excitation slit): 2.5/emi slit (emission slit): 2.5.
exi (wavelength of light for excitation): 554 nm/emi (wavelength of light for excitation): 579 nm.

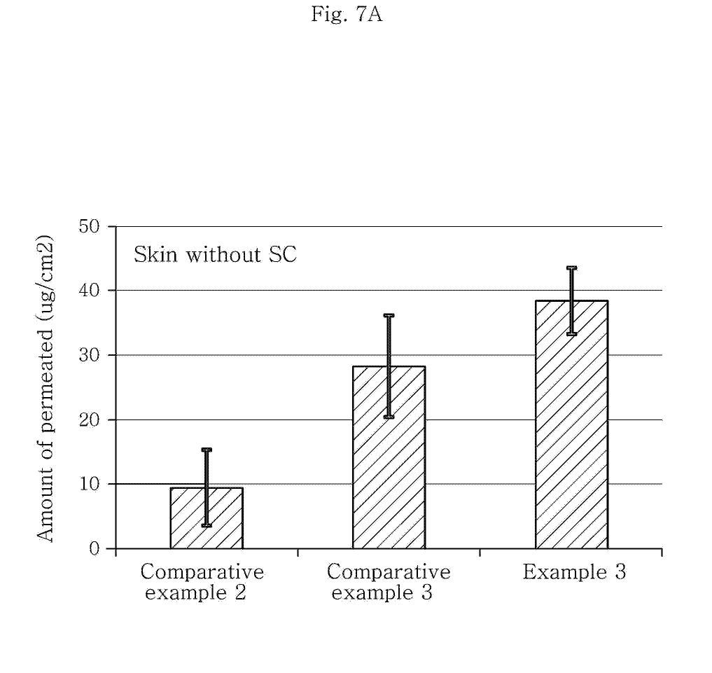

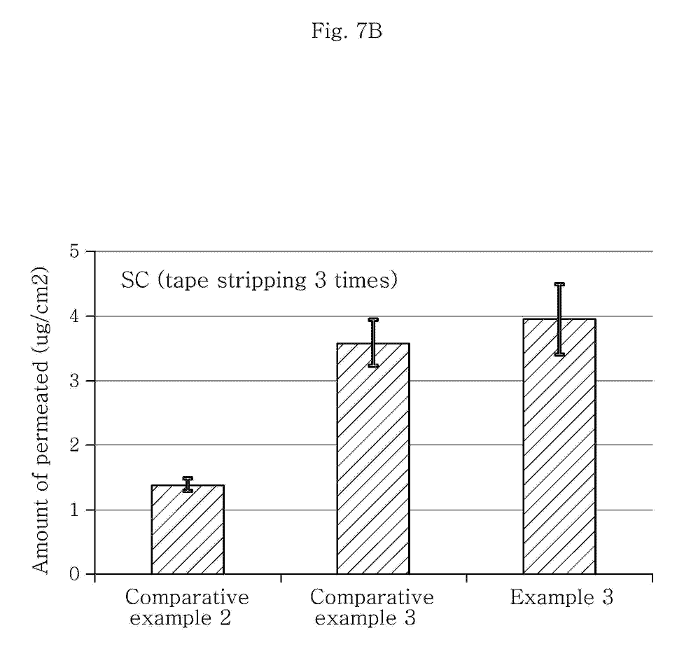

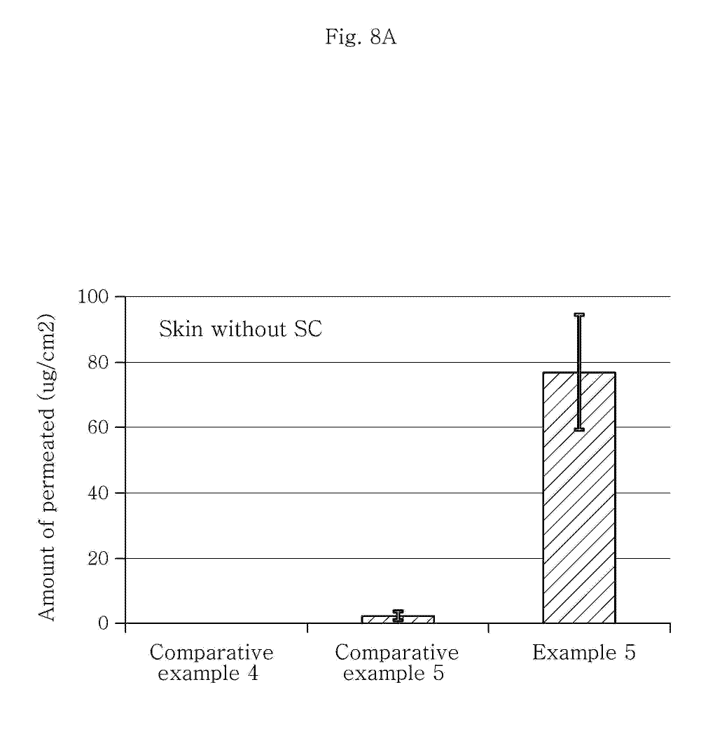

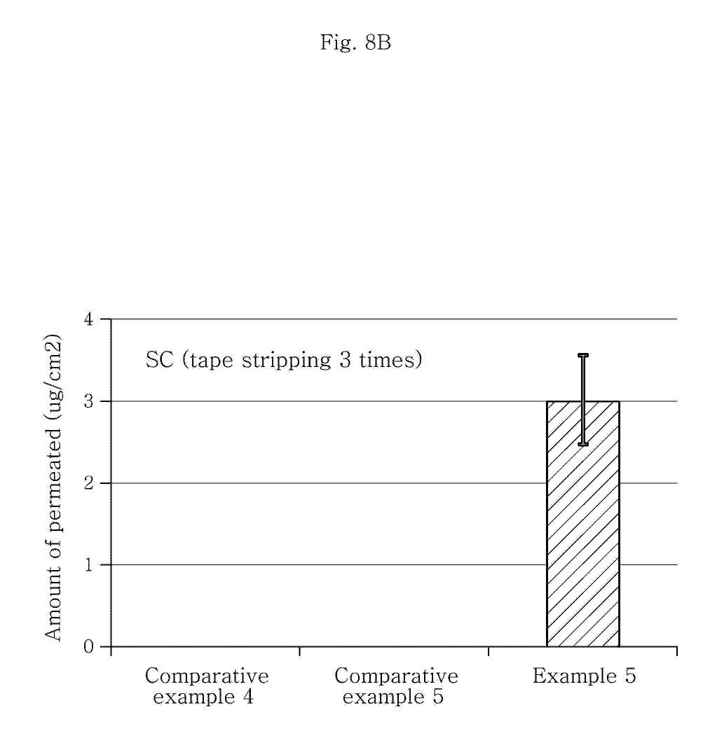

After excitation with light having a wavelength of 544 nm, the intensity of fluorescence emission from the fluorophore was detected at 579 nm. The quantitative result is shown in FIGS. 7 and 8 and Table 5. No fluorescence emission was observed form the receptor part.

TABLE 5

| | Average for | | Average for stratum | |
|---|---|---|---|---|
| Samples | skin (μg/cm²) | STD | corneum (μg/cm²) | STD |
| Comparative | 9.526 | 5.889 | 1.388 | 0.091 |
| Example 2 | | | | |
| Comparative | 28.319 | 7.931 | 3.588 | 0.361 |
| Example 3 | | | | |
| Example 3 | 38.447 | 5.134 | 3.957 | 0.544 |
| Comparative | 0.000 | — | 0.000 | — |
| Example 4 | | | | |
| Comparative | 2.516 | 1.356 | 0.000 | — |
| Example 5 | | | | |
| Example 5 | 77.014 | 17.673 | 3.025 | 0.548 |
| Example 7 | 5.735 | 3.492 | 0 | — |
| Example 8 | 38.251 | 8.562 | 2.019 | 0.478 |

From FIGS. 7 and 8 and Table 5, it can be seen that, for rhodamine B (Comparative Example 2, Comparative Example 3 and Example 3), the concentration of rhodamine B absorbed in the stratum corneum and the skin tissue excluding the stratum corneum was in the order of Comparative Example 2<Comparative Example 3<Example 3. Accordingly, it was confirmed that the liposome structure enhances transdermal absorption of a material having a small molecular weight.

A different behavior was observed for dextran-RITC (Comparative Example 4, Comparative Example 5 and Example 5). In the stratum corneum, the fluorophore was hardly absorbed for Comparative Example 4 or for Comparative Example 5 wherein the general liposome was used. In contrast, the drug delivery carrier according to an aspect of the present disclosure (Example 5) resulted in remarkably higher absorption than Comparative Examples 4 and 5. A remarkable absorption of the fluorophore was observed in the skin tissue excluding the stratum corneum for Example 5, unlike Comparative Examples 4 and 5. Accordingly, it was confirmed that the drug delivery carrier according to an aspect of the present disclosure exhibits an effect of increasing the absorption of a material having a large molecular weight in the stratum corneum and the skin tissue. This effect is remarkable and distinct as compared to that of the existing liposome.

In addition, it can be seen from the result of Example 7 and Example 8 that the drug delivery carrier according to an aspect of the present disclosure provides remarkably high transdermal absorption as compared to one not containing the amphiphilic polymer when mixed with a nanoemulsion-type formulation. Specifically, when the drug delivery carrier containing the amphiphilic polymer (Example 6) or the drug delivery carrier not containing the amphiphilic polymer (Example 5) was mixed with a nanoemulsion, transdermal absorption of the macromolecule physiologically active ingredient dextran-RITC was 7 times or greater when the amphiphilic polymer was contained (Example 8) as compared to Example 7. The enhanced transdermal absorption is thought to be due to the enhanced structural stability of the liposome. From this experimental result, it was confirmed that the drug delivery carrier having the amphiphilic polymer introduced has enhanced structural stability and is capable of delivering a macromolecule physiologically active ingredient well into skins when contained in an emulsion-type formulation commonly used in cosmetics.

What is claimed is:

1. A drug delivery carrier comprising a lipid structure or a polymer particle covalently bonded to a cell-penetrating peptide or a peptide chain comprising the same,
wherein
a physiologically active ingredient is encapsulated in the lipid structure or the polymer particle,
the physiologically active ingredient is a water-soluble or water-insoluble macromolecule having a number-average molecular weight or a weight-average molecular weight of 500 Da or greater, and
the cell-penetrating peptide is a peptide having a sequence of Gly (Arg)ₙ Gly Tyr Lys Cys (1≦n≦20).

2. The drug delivery carrier according to claim 1, wherein the water-soluble or water-insoluble macromolecule has a number-average molecular weight or a weight-average molecular weight of 5,000 Da or greater.

3. The drug delivery carrier according to claim 1, wherein the sequence is a sequence of SEQ ID NO 1 (Gly Arg Arg Arg Arg Arg Arg Arg Arg Arg Gly Tyr Lys Cys).

4. The drug delivery carrier according to claim 1, wherein the lipid structure or the polymer particle further comprises an amphiphilic polymer.

5. The drug delivery carrier according to claim 4, wherein the cell-penetrating peptide or the peptide chain comprising the same is covalently bonded to the amphiphilic polymer.

6. The drug delivery carrier according to claim 4, wherein the amphiphilic polymer is one or more selected from a group consisting of an alkylated hyaluronic acid having an alkyl group attached to a hyaluronic acid side chain, a poly(methacrylic acid-co-n-alkyl methacrylate) random copolymer of Chemical Formula 1 and a poly(hydroxyethyl methacrylate-co-n-alkyl methacrylate) random copolymer of Chemical Formula 2: 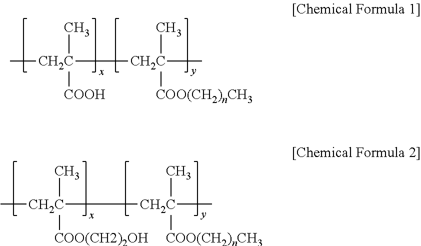
wherein
7≦n≦22, and
a molar ratio of x:y is from 90:10 to 50:50.

7. The drug delivery carrier according to claim 4, wherein the drug delivery carrier comprises 1-50 wt % of the amphiphilic polymer and 50-99 wt % of the lipid structure or the polymer particle based on the total weight of the lipid structure or the polymer particle and the amphiphilic polymer, and
wherein the amphiphilic polymer has a number-average molecular weight of 5,000-200,000 Da.

8. The drug delivery carrier according to claim 1, wherein the drug delivery carrier comprises a lipid structure and further comprises a cholesterol derivative.

9. The drug delivery carrier according to claim 8, wherein the lipid structure comprises one or more of dioleyl phosphatidylethanolamine, phosphatidylcholine and a distearoyl phosphatidylethanolamine-polyethylene glycol-maleimide (DSPE-PEG-Mal) composite as the lipid structure.

10. The drug delivery carrier according to claim 9, wherein the drug delivery carrier comprises dioleyl phosphatidylethanolamine, phosphatidylcholine, a cholesterol derivative and a distearoyl phosphatidylethanolamine-polyethylene glycol-maleimide (DSPE-PEG-Mal) composite at a molar ratio of 1.0-2.0:1.0-2.0:1.0-3.0:0.01-1.0.

11. The drug delivery carrier according to claim 1, wherein the lipid structure is a liposome or an emulsion and a lipid component of the liposome or the emulsion is a phospholipid or a nitrolipid having a C₁₂-C₂₄ fatty acid chain.

12. The drug delivery carrier according to claim 11, wherein the phospholipid is one or more selected from a group consisting of a natural phospholipid such as egg yolk lecithin (phosphatidylcholine), soy lecithin, lysolecithin, sphingomyelin, phosphatidic acid, phosphatidylserine, phosphatidylglycerol, phosphatidylinositol, phosphatidylethanolamine, diphosphatidylglycerol, cardiolipin and plasmalogen, a hydrogenation product obtainable from the natural phospholipid by a common method, a synthetic phospholipid such as dicetyl phosphate, distearoylphosphatidylcholine, distearoylphosphatidyl-ethanolamine (DSPE), dioleoylphosphatidylethanolamine, dipalmitoylphosphatidylcholine, dipalmitoylphosphatidylethanolamine, dipalmitoylphosphatidylserine, eleostearoylphosphatidylcholine, eleostearoylphosphatidylethanolamine and eleostearoylphosphatidylserine, and a fatty acid mixture obtainable from hydrolysis thereof.

13. The drug delivery carrier according to claim 12, wherein the phospholipid is a combination of phosphatidylcholine and phosphatidylethanolamine, a combination of phosphatidylcholine and phosphatidylglycerol, a combination of phosphatidylcholine and phosphatidylinositol, a combination of phosphatidylcholine and phosphatidic acid, a combination of phosphatidylcholine and dioleoylphosphatidylethanolamine or a combination of phosphatidylcholine, dioleoylphosphatidylethanolamine and phosphatidylserine.

14. The drug delivery carrier according to claim 13, wherein the combination is a combination of phosphatidylcholine, dioleoylphosphatidylethanolamine and phosphatidylserine, and the mixing ratio of the phosphatidylcholine, the dioleoylphosphatidylethanolamine and the phosphatidylserine is 1-4:1-2:1-2.

15. The drug delivery carrier according to claim 11, wherein the lipid component is contained in an amount of 0.001-20 wt % based on the total weight of the liposome suspension or emulsion.

16. The drug delivery carrier according to claim 1, wherein the polymer particle is an amphiphilic polymer or a biodegradable aliphatic polyester-based polymer, and the biodegradable aliphatic polyester-based polymer is one or more selected from a group consisting of poly(D,L-lactic acid), poly(L-lactic acid) or poly(D-lactic acid) of Chemical Formula 3, poly(D,L-lactic acid-co-glycolic acid), poly(D-lactic acid-co-glycolic acid) or poly(L-lactic acid-co-glycolic acid) of Chemical Formula 4, poly(caprolactone), poly(valerolactone), poly(hydroxybutyrate), poly(hydroxyvalerate), poly(1,4-dioxan-2-one), poly(orthoester) and a copolymer prepared from monomers thereof: 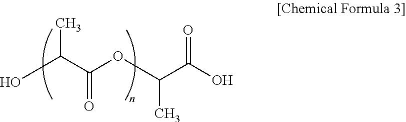
wherein n is an integer 2 or greater 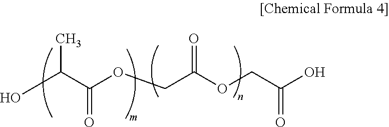
wherein each of m and n are, which may be identical or different, is an integer 2 or greater.

17. A composition comprising the drug delivery carrier according to claim 1 and a physiologically active ingredient encapsulated in the carrier.

18. The composition according to claim 17, wherein the physiologically active ingredient is one or more selected from a group consisting of a synthesized water-soluble macromolecular substance, a macromolecular substance extracted from a natural product, an enzyme, EGF, a protein, a peptide and a polysaccharide, and
wherein the amount of the physiologically active ingredient encapsulated in the drug delivery carrier is 0.01-30 wt % based on the total weight of the lipid structure or the polymer particle.

19. The composition according to claim 17, wherein the composition is a pharmaceutical composition in the form of a formulation selected from a formulation external application to skin, a formulation for oral administration and an injection.

20. The composition according to claim 17, wherein the composition is a cosmetic composition in the form of one or more formulation selected from a group consisting of a skin lotion, a skin softener, a skin toner, an astringent, a lotion, a milk lotion, a moisturizing lotion, a nourishing lotion, a massage cream, a nourishing cream, a moisturizing cream, a hand cream, a foundation, an essence, a nourishing essence, a pack, a soap, a cleansing foam, a cleansing lotion, a cleansing cream, a body lotion and a body cleanser.


SEQUENCE LISTING (3 sequences)

SEQ ID NO: 1 - protein, 14 aa, Artificial Sequence
  synthetic construct  AP-GRR peptide
GRRRRRRRRRGYKC

SEQ ID NO: 2 - protein, 15 aa, Drosophila melanogaster
RQIKIWFQNRMKWKK

SEQ ID NO: 3 - protein, 9 aa, Human immunodeficiency virus type 1
RKKRRQRRR